(19) United States
(12) Patent Application Publication
(10) Pub. No.: US 2015/0233903 A1
(43) Pub. Date: Aug. 20, 2015

(54) SOLID PHASE GLYCAN AND GLYCOPEPTIDE ANALYSIS AND MICROFLUIDIC CHIP FOR GLYCOMIC EXTRACTION, ANALYSIS AND METHODS FOR USING SAME

(71) Applicant: THE JOHNS HOPKINS UNIVERSITY, Baltimore, MD (US)

(72) Inventors: Hui Zhang, Ellicott City, MD (US); Shuang Yang, Ellicott City, MD (US); Punit Shah, Baltimore, MD (US); Shisheng Sun, Baltimore, MD (US)

(21) Appl. No.: 14/427,187

(22) PCT Filed: Sep. 10, 2013
(86) PCT No.: PCT/US2013/059038

§ 371 Date: Mar. 10, 2015

Related U.S. Application Data
(60) Provisional application No. 61/699,178, filed on Sep. 10, 2012.
(60) Provisional application No. 61/770,151, filed on Feb. 27, 2013.
(60) Provisional application No. 61/831,731, filed on Jun. 6, 2013.

(51) Int. Cl. G01N 33/53

(52) U.S. Cl. CPC G01N 33/5308; G01N 2400/00; G01N 2560/00

(57) ABSTRACT

Highly specific and novel solid phase methods for analyzing glycans and proteoglycans using a solid phase system are provided. The present invention also provides an integrated apparatus and methods of use which comprises a high-throughput glycan isolation and reverse-phase liquid chromatography (RPLC) for on-chip glycan extraction, modification and separation. The coverage of detected N-glycans by the GIG-chip-LC apparatus of the present invention can be significantly improved, especially for the low abundant species. Chip-LC by PGC minimizes dynamic range of glycan concentrations in fractions, resulting in detection of low-abundance glycans. Glycan isomers were able to be separated by the chip-LC portion of the apparatus. The GIG-chip-LC apparatus of the present invention can be used to analyze glycans from tissue and sera samples, thus providing a reliable tool for glycomic analysis. The reproducible performance and ability to detect unique glycans from tissue samples provides a powerful means for discovery of abnormal glycans associated with disease states.

### REFERENCE TO RELATED APPLICATIONS

This application claims the benefit of U.S. Provisional Patent Application Nos. 61/699,178 filed on Sep. 10, 2012, 61/770,151, filed on Feb. 27, 2013, and 61/831,731, filed on Jun. 6, 2013, all of which are hereby incorporated by reference for all purposes as if fully set forth herein.

### STATEMENT OF GOVERNMENTAL INTEREST

This invention was made with government support under grant nos. CA152813 and HL107153 awarded by the NIH. The government has certain rights in the invention.

### BACKGROUND OF THE INVENTION

Protein glycosylation is one of the most common and diverse protein modifications in which complex glycans are attached to glycoproteins. It is estimated that over 70% of all human proteins are glycosylated. Glycan biosynthesis relies on a great number of highly competitive processes involving glycosyltransferases and glycosidases, substrate availability, and the expression and structure of the glycosylated proteins and glycosylation sites. Therefore, protein glycosylation greatly depends on its biochemical environment. Aberrant glycosylation is believed to associate with the occurrence of many diseases such as cancers, inflammation, human immunodeficiency virus, and atherosclerosis, and thus glycomics analysis can contribute to the discovery of novel disease biomarkers or therapeutics. In addition, glycans affect protein stability, binding, and immunogenicity, and they play critical roles in developing glycoprotein therapeutics such as monoclonal antibodies. However, compared to genomics and proteomics, analytical techniques for glycomics lag far behind.

Several methods have been developed for glycan analysis. Typically, glycans are first released from glycoproteins or glycopeptides by enzymes such as Peptide: N-Glycosidase F (PNGase F) for N-linked glycans or by chemicals reactions, like β-elimination for O-linked glycans. Upon the release, glycans are desalted and purified from enzymes, chemicals, and their concatenate peptides for mass spectrometry analysis. Although glycans are purified by separating them from peptides and other non-glycan molecules by using a variety of methods such as affinity column, reverse-phase high-performance liquid chromatography, capillary electrophoresis, hydrophilic interaction chromatography, or multidimensional separations, the major obstacle for these methods is their incapability to separate glycans or glycopeptides from other species, especially from the non-glycosylated peptides. In terms of glycan purification, the graphite guard column is a widely used medium for glycan purification, mostly for the removal of salts and small molecules. However, the graphite column separates glycans and other molecules in the complex samples based on hydrophobicity; the column will also isolate the nonspecific hydrophilic species and the low molecular weight of peptides in the glycan fraction. As a result, the yield and specificity of glycans recovered from complex glycoprotein samples remain low.

Mass spectrometry is the emerging technology for analyzing quantitative glycan structures. However, identification and accurate quantification of glycans, especially sialylated glycans, is challenging. This is due to the fact that sialylated glycans have negative charges that have decreased ionization efficiency compared to neutral glycans. The labile nature of sialic acid also makes the analysis of glycans challenging due to the loss of sialic acids within glycans during MS analysis before the glycan ions reach the detector. There have been a few methods adopted to modify sialic acids in neutralizing and making glycan MS amicable. Permethylation is also a widely used method to stabilize the acidic component of glycans by modifying hydroxyl, amino, carbonyl and carboxylic moieties of glycans with methyl groups. This methyl incorporation makes the glycan ionization more efficient, and also prevents the loss of sialic acids. Other approaches are also reported to modify sialic acids including esterification, amidation, and oxime formation reactions.

Rapid isolation and separation of glycans from complex biological samples is crucial for glycomics analysis in order to analyze glycans by different instruments such as fluorescence spectroscopy and mass spectrometry (MS). While lectins can enrich glycans by affinity interactions, each lectin may be only effective on certain type of glycans so that it lacks capability for global glycan enrichment and subsequent glycan profiling.

Chromatographic methods such as size exclusion and hydrophilic columns are commonly used for glycan enrichment and separation. To increase the detectability and quantitation of glycan analysis, they, especially sialylated glycans, need further modifications. During this process, sample loss is inevitable. Solid-phase glycomics analysis via chemoselective approaches recently became popular for glycan isolation. For example, the hydrazide-functionalized beads react to aldehyde groups of reducing ends of glycans and other non-conjugated molecules are removed for purification before release of the glycans from beads via hydrazone hydrolysis.

While glycan isolation, modification, separation have been widely achieved by chromatographic approaches in which glycans are first deglycosylated from glycoproteins by enzymatic digestion, separated from proteins and purified by columns, and derivatized by permethylation, reducing end labeling, or sialic acid modification before LC-MS. These glycomic analysis procedures are time consuming and could cause sample loss during the multi-step sample handling.

Quantitative analysis of glycans from normal and disease specimens can provide insight into disease onset and progression. Glycan quantification usually requires modification of the glycans with either chromogenic or fluorogenic tags for optical measurement or isotopic tags for mass spectrometric analysis. Due to rapid advances in mass spectrometry instruments in resolution, sensitivity and speed, MS-based methods have become increasingly popular for glycan analysis in the past decade. However, current isotopic tags for glycan labeling are primarily limited to mass-difference tags, which generate mass differences in precursor ions for quantification, but can complicate mass spectrometry results by occupying the mass spectrum.

There still exists, therefore, an need to develop analytical methods which can specifically isolate glycans or glycopeptides, including sialylated glycans, from complex mixtures, as well as cost-effective and sensitive high-throughput methods for comprehensive analysis of glycans from biological or clinical samples.

### SUMMARY OF THE INVENTION

In accordance with an embodiment, the present invention provides a method of isolating glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) releasing the glycans from the glycoproteins and/or glycopeptides bound to the solid support of c); and g) isolating the glycans released from f).

In accordance with an embodiment, the present invention provides a method of analyzing glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) releasing the glycans from the glycoproteins and/or glycopeptides bound to the solid support of c); and g) isolating the glycans released from f); and h) analyzing the glycans of g).

In accordance with another embodiment, the present invention provides a method of isolating glycans in a biological sample comprising: a) obtaining a biological sample comprising glycoproteins; b) denaturing the sample of a) to denature the glycoproteins and/or glycopeptides; c) reacting the glycoproteins and/or glycopeptides of b) with guanidine to convert lysine to homoarginine; d) conjugating the denatured glycoproteins and/or glycopeptides from b) with reductive amination to aldehyde groups on solid support; e) blocking unreacted aldehyde groups on the solid support with reductive amination; f) labeling aspartic acid groups by aniline using isotopes; g) performing an Asp-N digest to remove any unlabeled aspartic acid residues; h) releasing the N-glycans from the glycoproteins and/or glycopeptides bound to the solid support of c) using PNGase F; i) digesting glycoproteins and/or glycopeptides on beads with Asp-N to release N-glycopeptides at the N-terminal of the glycosylation motif (▾NXT/S); and j) isolating the isolated glycans released from h).

In accordance with another embodiment, the present invention provides a method of analyzing glycans in a biological sample comprising: a) obtaining a biological sample comprising glycoproteins; b) denaturing the sample of a) to denature the glycoproteins and/or glycopeptides; c) reacting the glycoproteins and/or glycopeptides of b) with guanidine to convert lysine to homoarginine; d) conjugating the denatured glycoproteins and/or glycopeptides from b) with reductive amination to aldehyde groups on solid support; e) blocking unreacted aldehyde groups on the solid support with reductive amination; f) labeling aspartic acid groups by aniline using isotopes; g) performing an Asp-N digest to remove any unlabeled aspartic acid residues; h) releasing the N-glycans from the glycoproteins and/or glycopeptides bound to the solid support of c) using PNGase F; i) digesting glycoproteins and/or glycopeptides on beads with Asp-N to release N-glycopeptides at the N-terminal of the glycosylation motif (▾NXT/S); j) isolating the isolated glycans released from h); and j) analyzing the glycans of g).

In some embodiments, the present invention provides methods for stabilizing and labeling sialylated glycans comprising preparing amidated derivatives of the sialylated glycans with p-toluidine in the presence of N-(3-dimethylaminopropyl)-N-ethylcarbodiimide (EDC).

In an embodiment, the present invention provides a method for isolating sialylated glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivatizing denatured glycoproteins and/or glycopeptides of c) with p-toluidine; g) releasing the glycans from the glycoproteins and/or glycopeptides bound to the solid support of c); and h) isolating the glycans released from g).

In another embodiment, the present invention provides a method for analyzing sialylated glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivatizing denatured glycoproteins and/or glycopeptides of c) with p-toluidine; g) releasing the glycans from the glycoproteins and/or glycopeptides bound to the solid support of c); h) isolating the glycans released from g); and i) analyzing the glycans of h).

In a further embodiment, the present invention provides a method for determining the number of sialic acid residues on isolated sialylated glycans in a biological sample comprising: a) obtaining a biological sample comprising glycoproteins; b) denaturing the sample of a) to denature the glycoproteins and/or glycopeptides; b1) dividing the denatured sample of b) into two or more aliquots; c) conjugating each aliquot of the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivitizing at least one aliquot of the denatured glycoproteins and/or glycopeptides of c) with light p-toluidine, and derivitizing at least one other aliquot of the denatured glycoproteins and/or glycopeptides of c) with heavy p-toluidine; g) releasing the glycans from each aliquot of the glycoproteins and/or glycopeptides bound to the solid support of c); and h) isolating the glycans released from each aliquot of g).

In yet another embodiment, the present invention provides a method for determining the number of sialic acid residues on isolated sialylated glycans in a biological sample comprising: a) obtaining a biological sample comprising glycoproteins; b) denaturing the sample of a) to denature the glycoproteins and/or glycopeptides; b1) dividing the denatured sample of b) into two or more aliquots; c) conjugating each aliquot of the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivitizing at least one aliquot of the denatured glycoproteins and/or glycopeptides of c) with light p-toluidine, and derivitizing at least one other aliquot of the denatured glycoproteins and/or glycopeptides of c) with heavy p-toluidine; g) releasing the glycans from each aliquot of the glycoproteins and/or glycopeptides bound to the solid support of c); h) isolating the glycans released from each aliquot of g); and i) analyzing the glycans of h).

In some embodiments, the present invention provides a method for preparing a library of glycans or glycopeptides from a sample comprising obtaining a sample from a subject and analyzing the glycans or glycopeptides in the sample using the methods described above.

In other embodiments, the present invention provides methods for preparing a glycan profile from a sample comprising obtaining a sample from a subject and analyzing the glycans in the sample using the methods described above to create a glycan profile.

In an embodiment, the present invention provides an apparatus for analysis of glycans in a sample comprising: a) a substrate in the form of a chip having at least a first and second layer, wherein the first layer is a fluid layer having at least a first and second channel, each channel having an inlet and an outlet and wherein each of the channels having a separation portion and a constrained portion, and wherein the first channel comprises a stationary phase for liquid chromatographic separation of glycans, and wherein the second channel comprises an aldehyde activated agarose bead resin, the outlet of the second channel intersects with the first channel and communicates with the first channel at a position proximal to the inlet of the first channel; b) the second layer is a coverslip layer which is fitted over top of the fluid layer and has at least three reservoirs, each having a removable cap which closes access to the inlet or outlet, wherein the first reservoir communicates with inlet of the first channel, the second reservoir communicates with inlet of the second channel, and the third reservoir communicates with outlet of the first channel; and c) the second layer is bonded to the first layer to make a liquid seal.

In another embodiment, the present invention provides a method for analysis of glycans in a sample comprising: a) injecting a sample containing glycans into the inlet of the second channel of the apparatus of the present invention with the inlet of the first channel open and the outlet of the first channel closed; b) allowing any proteins in the sample to conjugate to the aldehyde activated agarose bead resin in the second channel; c) reducing the conjugated proteins with a reducing reagent and blocking any free aldehyde groups on the resin in the second channel; d) washing the second channel with water; e) releasing the glycans with a releasing agent in the second channel; f) flushing the glycans via the outlet of the second channel into the first channel at a position proximal to the inlet of the first channel; g) closing the inlet of the second channel and pumping mobile phase into the inlet of the first channel while collecting eluent from the outlet of the first channel; and h) analyzing the glycans in the eluent.

In a further embodiment, the present invention provides a method for preparing a library of glycans or glycopeptides from a sample comprising obtaining a sample from a subject and analyzing the glycans or glycopeptides in the sample using the apparatus and/or methods of the present invention, to create a glycan library.

In still another embodiment, the present invention provides a method for preparing a glycan profile from a sample comprising obtaining a sample from a subject and analyzing the glycans in the sample using the methods described above to create a glycan profile.

In yet a further embodiment, the present invention provides the use of a glycan or glycopeptide profile prepared using the methods described above, to diagnose a disease or condition in a subject comprising comparing the glycan or glycopeptide profile from a subject to a glycan profile from a normal sample or diseased sample and determining whether the sample of the subject has the disease or condition.

### BRIEF DESCRIPTION OF THE DRAWINGS

FIG. 1 is a schematic diagram of glycan capture and release using solid-phase glycan extraction (SPGE) method.

FIG. 2 is an analysis of glycans from SGP using SPGE and modification of glycan on the solid support. The mass spectra for the SGP glycopeptide (m/z=2865.8 Da) before (A) and after (B) a 6-hour conjugation reaction to the solid support. The released glycan from SGP peptide before (C) and after (D) neuraminidase treatment.

FIG. 3 is mass spectra of the extracted high mannose glycans from 1 μg RNase B isolated using the SPGE method. All five previously reported mannose glycans of RNase B were observed.

FIG. 4. Glycan analysis from human serum using SPGE.

FIG. 5. MALDI-MS spectra of glycans extracted from four prostate cancer cell lines using SPGE method in low mass range (top) and high mass range (bottom).

FIG. 6. Analysis of glycoproteins on four prostate cancer cell lines using AAL lectin (A), ConA lectin (B), and protein staining (C).

FIG. 7 is a schematic of the solid-phase extraction of glycopeptides and glycans (SPEGAG) method of the present invention.

FIG. 8A shows a schematic of an alternate embodiment of the SPEGAG method of the present invention.

FIG. 8B is a MALDI-MS spectra of N-glycopeptides and N-glycans from fetuin were isolated using the method of FIG. 8A.

FIG. 9 is one MS/MS spectrum of N-glycopeptides N^{#}CSVRQQTQHAVEG.D from bovine fetuin using the alternative solid-phase extraction of glycopeptides and glycans (SPEGAG) method.

FIG. 10 depicts the scheme strategy for the solid-phase labeling of sialic acid and quantitative analysis of sialylated glycans by mass spectrometry. Proteins are conjugated to solid support. Bound proteins are labeled with light or heavy p-toluidine. N-glycans are released from the proteins using PNGase F. N-glycans are analyzed using MALDI-MS.

FIG. 11 shows a mass spectrum from Shimadzu AXIMA Resonance MALDI Mass spectrometer of Nglycans from bovine fetuin. Sialic acids of N-glycans were amidated with p-toluidine in presence of EDC. Glycans were analyzed in DHB/DMA matrix by positive ion mode. 1000 shots were acquired. Glycoworkbench was used for illustrations of the most plausible structure based on accurate mass. Dark square represents GlcNAc, dark grey circle represents mannose, light grey circle represents galactose, dark triangle represents fucose and light diamond represents p-toluidine modified sialic acid.

FIG. 12 depicts a MALDI-MS spectrum of N-glycans from human serum. Two equal aliquots of serum proteins were bound to beads and one aliquot was labeled with light p-toluidine, second aliquot was labeled with heavy p-toluidine D9. N-glycans were released using PNGase F. The mixture of glycans was subjected to MALDI-MS analysis. 12A) Light labeled monosialylated N-glycan. 12B) Heavy labeled mono sialylated N-glycan. 12C) MS spectrum of 1:1 mixture of light and heavy labeled monosialylated glycan. 12D) Mass spectrum of serum N-glycans, difference between the peak pairs from light and heavy labeled sialic acids represents number of sialic acids present in the N-glycan structure. Glycoworkbench was used for cartoons of the most plausible structure based on accurate mass and difference between the doublets.

FIG. 13 shows N-Linked glycans of proteins from pancreatic cancer cell line SW1990 treated with 1,3,4-O-Bu3ManNAc. 13A) Mass spectrum of N-glycans from SW1990 cells treated with 1,3,4-O-Bu3ManNAc and labeled with heavy p-toluidine. 13B) Mass spectrum of N-glycans from SW1990 cells not treated with 1,3,4-O-Bu3ManNAc as control and labeled with light p-toluidine. 13C) Mass Spectrum of N-glycans from SW1990 cells treated with and without 1,3,4-O-Bu3ManNAc and labeled with heavy and light p-toluidine respectively and then mixed in 1:1 ratio.

FIG. 14 depicts a mass spectrum from Shimadzu AXIMA Resonance MALDI Mass spectrometer of unmodified N-glycans from bovine fetuin. Glycans were analyzed in DHB/DMA matrix by positive ion mode. 1000 shots were acquired.

FIG. 15 shows Mass spectrum from Shimadzu AXIMA Resonance MALDI Mass spectrometer of N-glycans from bovine fetuin. Sialic acids of N-glycans were amidated with acetohydrazide in presence of EDC. Glycans were analyzed in DHB/DMA matrix by positive ion mode. 1000 shots were acquired.

FIG. 16 depicts Mass spectrum from Shimadzu AXIMA Resonance MALDI Mass spectrometer of N-glycans from bovine fetuin. Sialic acids of N-glycans were amidated with aniline in presence of EDC. Glycans were analyzed in DHB/DMA matrix by positive ion mode. 1000 shots were acquired.

FIG. 17 illustrates an embodiment of the present invention. (1A) A schematic diagram of the fluid layer of the GIG-chip-LC apparatus for glycan capture and separation, and the locations of A, B and C for reservoirs and needle insertion. (1B) A schematic diagram depicting the coverslip layer of the apparatus and the ports A-C with needles.

FIG. 18 is a schematic diagram depicting the operation of an embodiment of the GIG-chip-LC apparatus of the present invention for glycan analysis. (A) Capture of proteins by infusing the proteins into the Aminolink bead-packed second channel. (B) Separation of released glycans in porous graphitized carbon particles in the first channel.

FIG. 19 shows N-glycans from mouse serum or heart tissue by separation with the GIG portion of the apparatus without use of the chip-LC portion. Mouse blood serum (MBS) (A) and mouse heart tissue (MT) (B) were analyzed by GIG and MS. The number representation of several N-glycans giving in Table 6, where Man5-Man9 are oligomannoses.

FIG. 20 depicts the profiling of N-glycans from mouse serum or heart tissue by use of the GIG-chip-LC apparatus and methods of the present invention. Mouse blood serum (MBS) (A) and mouse heart tissue (MT) (B) were analyzed by GIG-chip-LC and MS. Oligomannoses were present in two fractions with acetonitrile concentrations at 21% and 22%; sialylated glycans were detected in eluents from 25% to 28% and 30% to 35%; triantennary and quadantennary sialylated glycans were detected in 40% and above.

FIG. 21 shows N-glycan coverage from human serum. A total of 65 N-glycan masses were detected from human serum using the GIG portion of the chip without LC fractionation (A). A total of 148 N-glycan masses were detected from human serum using the GIG-chip-LC together (B). The extracted N-glycans from human serum using GIG was directly analyzed by MALDI-MS.

FIG. 22 shows N-glycans isolated from ribonuclease B by GIG-microchip. Five oligomannose glycans were eluted from GIG portion of the microchip and detected by MALDI-MS.

FIG. 23 depicts reproducible N-glycan fractionation by GIG-chipLC. N-glycans of mouse blood serum (MBS). (A) 400 μg of serum proteins; (B) 200 μg of serum proteins were extracted using the GIG-chip-LC apparatus and methods. Fractionated N-glycans were detected by Shimadzu AXIMA Resonance MALDI-MS.

### DETAILED DESCRIPTION OF THE INVENTION

Methods are provided herein which are directed to improved methods of analyzing carbohydrates. As used herein, the term “carbohydrate” is intended to include any of a class of aldehyde or ketone derivatives of polyhydric alcohols. Therefore, carbohydrates include starches, celluloses, gums and saccharides. Although, for illustration, the term “saccharide” or “glycan” is used below, this is not intended to be limiting. It is intended that the methods provided herein can be directed to any carbohydrate, and the use of a specific carbohydrate is not meant to be limiting to that carbohydrate only.

As used herein, the term “saccharide” refers to a polymer comprising one or more monosaccharide groups. Saccharides, therefore, include mono-, di-, tri- and polysaccharides (or glycans). Glycans can be branched or branched. Glycans can be found covalently linked to non-saccharide moieties, such as lipids or proteins (as a glycoconjugate). These covalent conjugates include glycoproteins, glycopeptides, peptidoglycans, proteoglycans, glycolipids and lipopolysaccharides. The use of any one of these terms also is not intended to be limiting as the description is provided for illustrative purposes. In addition to the glycans being found as part of a glycoconjugate, the glycans can also be in free form (i.e., separate from and not associated with another moiety). The use of the term peptide is not intended to be limiting. The methods provided herein are also intended to include proteins where “peptide” is recited.

Herein is described novel methods for the high-throughput analysis of glycans conjugated to proteins from complex samples using Solid-Phase Glycan Extraction (SPGE). Glycoproteins or glycopeptides were immobilized on a solid support, other molecules were removed, and glycans were then released from glycoproteins/glycopeptides for analysis by mass spectroscopy. The methods of the present invention were applied to the analysis of glycans from human serum and cells. The extracted glycans from four types of cancer cells indicated that fucosylated and high mannose glycans are differentially expressed in androgen-independent cells.

SPGE has a number of advantages for glycan analysis. Without be limited to any particular example, specific glycans were directly analyzed without further purification. This allows for high yields from the glycan isolation and high sensitivity of detection and also reduced time and cost by eliminating traditional glycan purification steps using C-18 and graphite columns. In addition, the solid-phase capture method provides a platform for glycan modifications using enzymes or chemicals. In the present invention, the inventors showed that exoglycosidase digestion was efficient while the glycosylated proteins were bound to the solid support. The enzymes and chemicals can be easily removed and other reagents added providing a specific and rapid method for glycan modification or derivatization from complex samples. Thus, the inventions provide a platform for glycan sequencing or targeted glycan synthesis using combination of chemicals and enzymes. Moreover, the SPGE procedure is quantitative and the isolated glycans are compatible with current downstream analytical platforms. In the methods of the present invention, the isolated glycans were analyzed by MS using label-free quantification, although the method can be used with stable-isotopic labeling of glycans to obtain accurate quantitation. Glycans can also be labeled with fluorescence tags for chromatography or electrophoresis analyses. The glycan captured from SGP by SPGE was tested. The CV of the repeated analyses was 12.87%, indicating that the glycan isolation using SPGE was quantitative.

In accordance with an embodiment, the present invention provides a method of isolating glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) releasing the glycans from the glycoproteins and/or glycopeptides bound to the solid support of c); and g) isolating the glycans released from f).

In another embodiment, the isolated glycans are subjected to an analysis step.

In some of the inventive methods, both N-linked and O-linked glycoproteins are conjugated to the solid support through reductive amination. It was demonstrated that N-glycans were specifically released from the solid support by PNGase F. After releasing N-glycans, O-glycans can be release from beads for analysis. However, there is no enzyme comparable to PNGase F for removing intact O-linked glycans. To successfully release O-linked oligosaccharides, it is necessary to sequentially remove monosaccharides by using a panel of exoglycosidases until only the Galβ1,3GalNAc core remains attached to the serine or threonine residue. The core can then be released by O-glycosidase. Since not all O-linked oligosaccharides contain this core structure, a chemical method, such as β-elimination may be more general and effective for the release of the formerly O-linked glycosylated peptides. The solid-phase capture of proteins also provides a powerful platform to study other protein post-translational modifications, such as acylation, phosphorylation, and ubiquitination.

In accordance with another embodiment, the present invention provides a method of isolating glycans in a biological sample comprising: the present invention provides a method of analyzing glycans in a biological sample comprising: a) obtaining a biological sample comprising glycoproteins; b) denaturing the sample of a) to denature the glycoproteins and/or glycopeptides; c) reacting the glycoproteins and/or glycopeptides of b) with guanidine to convert lysine to homoarginine; d) conjugating the denatured glycoproteins and/or glycopeptides from b) with reductive amination to aldehyde groups on solid support; e) blocking unreacted aldehyde groups on the solid support with reductive amination; f) labeling aspartic acid groups by aniline using isotopes or iART ligands; g) performing an Asp-N digest to remove any unlabeled aspartic acid residues; h) releasing the N-glycans from the glycoproteins and/or glycopeptides bound to the solid support of c) using PNGase F; i) digesting glycoproteins and/or glycopeptides on beads with Asp-N to release N-glycopeptides at the N-terminal of the glycosylation motif (▾NXT/S); j) isolating the isolated glycans released from h); and j) analyzing the glycans of g). In another embodiment, the isolated glycans are subjected to an analysis step.

In accordance with an embodiment, the present invention provides the derivitization of sialylated glycans via amidation with p-toluidine to stabilize the sialic acid glycans. The p-toluidine also neutralizes negatively charged sialic acids and renders derivatized N-glycans more hydrophobic for mass spectrometry detection. In some embodiments, proteins are first conjugated to solid support by reduction amination. The sialic acid groups on conjugated proteins are then modified by adding p-toluidine in the presence of EDC. Glycans are then released from proteins on solid support and analyzed by mass spectrometry in positive mode. When light and stable isotopic heavy p-toluidine reagents are used in derivatization reaction, seven mass unit differences between light and heavy tags for each sialic acid are effectively resolved in the mass spectrum, allowing the identification of number of sialic acids in the glycan structures or relative quantitation of sialylated glycans from different samples. While not being limited to any particular example, the methods of the present invention were applied to the identification of sialylated N-glycans from fetuin and human serum and also used for the quantitative analysis of sialylated glycans from pancreatic cancer cells, SW1990, to study N-glycan sialylation after the cells were incubated with a ManNAc analog to increase metabolic flux through the sialic acid biosynthetic pathway.

In an embodiment, the present invention provides a method for isolating sialylated glycans in a biological sample comprising: a) obtaining a biological sample comprising glycoproteins; b) denaturing the sample of a) to denature the glycoproteins and/or glycopeptides; c) conjugating the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivitizing denatured glycoproteins and/or glycopeptides of c) with p-toluidine; g) releasing the glycans from the glycoproteins and/or glycopeptides bound to the solid support of c); and h) isolating the glycans released from g). In another embodiment, the isolated glycans are subjected to an analysis step.

The methods of the present invention also provide for identifying the number of sialic acid residues on a particular isolated glycan. In a further embodiment, the present invention provides a method for determining the number of sialic acid residues on isolated sialylated glycans in a biological sample comprising: a) obtaining a biological sample comprising glycoproteins; b) denaturing the sample of a) to denature the glycoproteins and/or glycopeptides; b1) dividing the denatured sample of b) into two or more aliquots; c) conjugating each aliquot of the denatured glycoproteins and/or glycopeptides from b) with reductive amination to aldehyde groups on solid support; d) blocking unreacted aldehyde groups on the solid support with reductive amination; e) removing unconjugated glycoproteins and/or glycopeptides and remaining components; f) derivitizing at least one aliquot of the denatured glycoproteins and/or glycopeptides of c) with light p-toluidine, and derivitizing at least one other aliquot of the denatured glycoproteins and/or glycopeptides of c) with heavy p-toluidine; g) releasing the glycans from each aliquot of the glycoproteins and/or glycopeptides bound to the solid support of c); and h) isolating the glycans released from each aliquot of g). In another embodiment, the isolated glycans are subjected to an analysis step.

In accordance with another embodiment of the present invention, it will be understood that the term “biological sample” or “biological fluid” includes, but is not limited to, any quantity of a substance from a living or formerly living subject. Such substances include, but are not limited to, blood, serum, plasma, urine, cells, organs, tissues, bone, bone marrow, lymph, lymph nodes, synovial tissue, CSF, chondrocytes, synovial macrophages, endothelial cells, and skin. In a preferred embodiment, the fluid is blood or serum.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

It will be understood by those of skill in the art that the denaturation of the glycoproteins in the sample, in the inventive methods, can be accomplished using any means known in the art. In addition to heating, denaturation agents and proteolysis may also be used. A “denaturing agent” is an agent that alters the structure of a molecule, such as a protein. Denaturing agents, therefore, include agents that cause a molecule, such as a protein to unfold. Denaturing can be accomplished, for instance, with heat, with heat denaturation in the presence of β-mercaptoethanol and/or SDS, by reduction followed by carboxymethylation (or alkylation), etc. Reduction can be accomplished with reducing agent, such as, dithiothreitol (DTT). Carboxymethylation or alkylation can be accomplished with, for example, iodoacetic acid or iodoacetamide. Denaturation can, for example, be accomplished by reducing with DTT, β-mercaptoethanol or tri(2-carboxyethyl)phosphine (TCEP) followed by carboxymethylation with iodoacetic acid. When the glycoconjugate sample is a sample of a body fluid, such as serum, the denaturation can be accomplished with EndoF. The glycoconjugates can also be denatured with denaturing agents, such as detergent, urea or guanidium hydrochloride.

In accordance with an embodiment, the methods of analyzing glycans of the present invention includes cleaving the glycans from the glycoconjugates using any chemical or enzymatic methods or combinations thereof that are known in the art. An example of a chemical method for cleaving glycans from glycoconjugates is hydrazinolysis or alkali borohydrate. Enyzmatic methods include methods that are specific to N- or O-linked sugars. These enzymatic methods include the use of Endoglycosidase H (Endo H), Endoglycosidase F (EndoF), N-Glycanase F (PNGaseF) or combinations thereof. In some preferred embodiments, PNGaseF is used when the release of N-glycans is desired. When PNGaseF is used for glycan release the proteins is, for example, first unfolded prior to the use of the enzyme. The unfolding of the protein can be accomplished with any of the denaturing agents provided above.

In accordance with an embodiment of the above methods of the present invention the denaturing of the sample in a) comprises: i) heating the sample for a sufficient period of time; ii) incubating the sample from i) with a proteolytic enzyme for a period of time; and iii) adding a sufficient amount of PNGase F to the sample of ii) to release the glycans from the peptide fragments.

It is understood by those of skill in the art that the proteolytic enzyme used in the inventive methods can be any enzyme capable of cleaving peptide bonds. Examples of proteolytic enzymes useful in the inventive methods include trypsin, chymotrypsin, papain, and pepsin.

After protein denaturation and/or digestion, the (If using Endo H, the peptide portion still containing carbohydrate) glycopeptides and glycoprotein fragments can be removed by washing or use of various column based methods known in the art. Alternatively, the glycopeptides and glycoprotein fragments can be collected and separately analyzed using known methods.

In accordance with an embodiment, the methods of the present invention include conjugating the free glycans in sample or the released glycans from glycoconjugates to a solid support. In an embodiment, conjugation of the free glycans or the released glycans of the sample in b) comprises: i) adding at least a portion of the sample from b) to a solid support comprising superparamagnetic hydrazide nanoparticles; ii) mixing the mixture of i); and iii) incubating the mixture of ii) for a sufficient time at a temperature of between 40-60° C. This allows the released glycans to form hydrazone bonds to the hydrazide moieties on the nanoparticles. In another embodiment, the hydrazide moieties could be ligated or otherwise chemically bound to any known solid support.

In accordance with a further embodiment, the conjugation of the released glycans to the solid support is performed in the absence or presence of a catalyst. It will be understood by those of skill in the art that catalysts suitable for use with the methods of the present invention will include those compounds that can act as a Schiff-base intermediate in the reaction of the free reducing ends of the glycans with the hydrazide moieties on the solid support. In the inventive methods, the catalyst can be added to the mixture of the released glycans and solid support. In accordance with an embodiment, the catalyst used in the inventive methods is aniline.

The solid substrate used to bind the glycans and glycopeptides in the inventive methods may be a material that may be modified to contain discrete individual sites appropriate for the attachment or association of the glycans and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics.

In an alternative embodiment, other supports, such as slides, for example can be used as the solid support. This is particularly useful for glycan analysis with spatial information and can be applied to glycan imaging of tissues. In yet another embodiment tags can also be used in place of solid supports, using well known ligands such as biotin-hydrazide or azido-hydrazide, that can be used to conjugate the glycans in solution and then are subsequently captured using the tags. This solution capture embodiment is particularly useful to capture glycans in vivo.

After the removal of the non-conjugated components, the glycans or glycopeptides (both N- and O-glycopeptides) can be released from beads by hydrolysis and analyzed. In accordance with an embodiment, the hydrolysis of the hydrazone bonds is accomplished by lowering the pH of the solution to <3. In some embodiments the range of pH is between about pH 1 to about pH 3, preferably about pH 2. While it will be understood that any acid solution can be used to accomplish this, such as, for example trifluoroacetic acid (<1% v/v), 0.01 M HCl, or 0.005 M H₂SO₄. In accordance with an embodiment, 10% v/v formic acid is suitable for this use.

As disclosed herein, the present invention provides an integrated apparatus for conducting glycomic capture, derivitization, and analysis of complex biological samples. The apparatus can be in the form of a microfluidic chip. The capture and derivitization of the glycans in the sample is performed in a portion of the apparatus which utilizes a chemoselective technique known as “glycoprotein immobilization for glycan extraction” or (GIG) which was developed by the present inventors and is described in detail in U.S. Patent Application Nos. 61/699,178 and 61/770,151, and are both incorporated by reference herein.

The apparatus or chip of the present invention also comprises a separation portion which encompasses microfluidic liquid chromatographic separation methods to separate the extracted glycans for further analysis. This separation portion is termed “chip-LC” as it is based on microfluidic chip technology. Thus, in one or more embodiments, the at least two portions, the GIG portion and the chip-LC portion, comprise the apparatus of the present invention.

In an embodiment, the apparatus comprises a polymer substrate in the form of a chip having a plurality of layers. In one embodiment, the chip comprises a fluid layer and a coverslip layer, which are fused together at final assembly of the apparatus. The fluid layer is composed of a plurality of channels having an inlet and an outlet. The channels are fabricated such that a portion of the channel proximal to the outlet end, termed “the constrained channel” has a smaller depth than the remainder of the channel, termed the “separation portion” of each channel.

In an embodiment, the fluid layer of the apparatus of the present invention comprises at least two channels, each having a separation portion and a constrained portion. The separation portion can have any dimension within the limits of the depth of the substrate. In an embodiment, the separation portion of the channels has dimensions of at least 500 μm×500 μm to 1000 μm×1000 μm, in a preferred embodiment, the separation portion of the channels has dimensions of about 800 μm×800 μm. The constrained portion of the channels has smaller dimensions to act as a weir, or virtual valve, to the stationary phase or separation substrate or support, which is disposed within the separation portion of the channels. In an embodiment, the constrained portion of the channels has dimensions of between about 25 μm×25 μm to about 100 μm×100 μm, in a preferred embodiment, the constrained portion of the channels has dimensions of about 50 μm×50 μm.

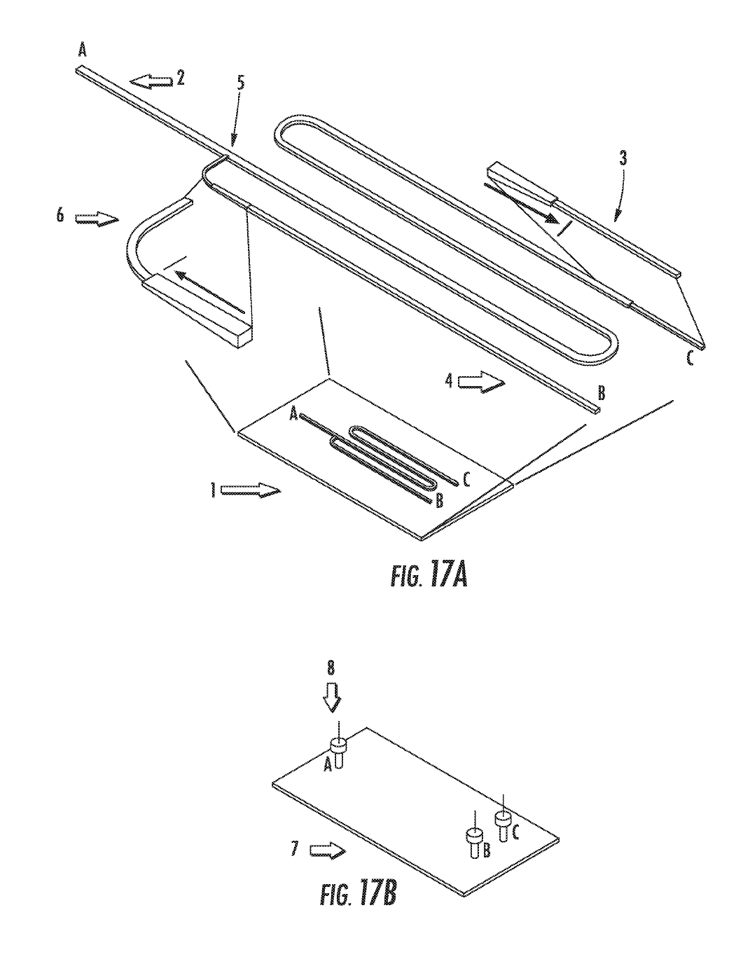

In an embodiment, a first channel ((2) in FIG. 17) has disposed within the separation portion, a compound which acts as a stationary phase for liquid chromatographic separation of glycans. This first channel is the “chip-LC” portion of the apparatus of the present invention. In one embodiment, the compound is porous graphitized carbon in particle form. A second channel ((6) in FIG. 17) has disposed within the separation portion, a compound which acts as an immobilization and reaction substrate for glycan capture, derivitization and separation. This second channel is the “GIG” portion of the apparatus of the present invention. In one embodiment, the compound in the separation portion of the second channel is an aldehyde activated agarose bead resin. In an embodiment, the resin is AminoLink resin (Pierce, Rockford, Ill.).

Referring now to FIG. 17, which depicts an embodiment of the fluid portion of the chip of the present invention in an exploded view, the fluid layer of the chip (1) is composed of a polymer substrate which has been milled to create a first channel (2) which has a first end or port (A) which acts both as an inlet or outlet, and a second end or port (C) which also acts both as an inlet or outlet. The constrained portion of the first channel (3) is proximal to port (C). The remainder of the first channel is the separation portion of the first channel. The polymer substrate is also milled to create a second channel (4) which has a first end or port (B) which acts both as an inlet or outlet, and a second end (5) which acts as an outlet into the separation portion of the first channel proximal to the first end of the first channel. The second channel also has a constrained portion (6) which is proximal to the second end of the second channel. The remainder of the second channel is the separation portion of the second channel.

Referring now to FIG. 17B, which depicts an embodiment of the coverslip layer of the apparatus. In an embodiment, the coverslip layer (7) is formed to match the dimensions of the fluid layer of the apparatus and to fit over top of the fluid layer. The coverslip layer (7) of the chip is also composed of a polymer, and has three reservoirs (8) drilled into it (A, B, C). The reservoirs communicate with the ends of the channels as shown in FIG. 17A. The diameters of the reservoirs are such that a tube of a specific diameter can pass through the coverslip layer to deliver liquid to the end of the channels in the fluid layer of the chip. In an embodiment, the diameters of the reservoirs are sufficient to allow a 22 gauge surgical needle to fit securely in each reservoir, and to act as an inlet or outlet to the channels in the fluid layer. The two layers are bonded together using known methods to provide a liquid seal. The flow direction in the channels of the apparatus is controlled by capping the appropriate needle port (A, B or C).

The ports A-C can be connected to any type of suitable external components i.e. syringe pump, syringe needle, and elution collectors, for example.

In an embodiment, the operation of the apparatus is as follows. To begin a sample analysis, port B is capped with ports A and C open, and the porous graphitized carbon (PGC) in the chip-LC portion of the apparatus and mobile phase, for example, 80% acetonitrile (0.1% FA) was flushed through the first channel from port A to port C, followed by additional flushing with a 0.1% FA washing solution. Port C is then capped and with port A and B open, and a sample is injected into the second channel of the GIG portion of the apparatus. The sample is allowed to interact with the aldehyde activated agarose bead resin for a sufficient period of time, for example, about 30 minutes to 3 hours, preferably about 2 hours. This allows the proteins to conjugate to the beads. The proteins in the sample are then reduced by injection of a reduction solution, such as a NaCNBH₃ solution, for between about 1 to 3 hours, preferably about 2 hours, from B to A. Any free aldehyde groups are then blocked by addition of reduction solution and Tris buffer. In order to modify sialized glycans, a solution of p-toluidine is then injected and allowed to incubate in the second channel with the resin for between 2 to 4 hours, preferably about 3 hours. The second channel is then washed from port B to port A with water. To release the glycans from the bound proteins on the resin, PNGase F was injected into the second channel at port B and allowed to incubate for about 1 to 3 hours, preferably about 2 hours. Released glycans are then loaded into the chip-LC portion of the apparatus by injection of washing solution into the second channel at port B. This allows the released glycans to move to the first channel and collect at the intersection of second end (5) of the second channel and the separation portion of the first channel.

To further separate the glycans using the chip-LC portion of the apparatus, port B is capped and ports A and C are opened. Mobile phase is then pumped into port A and eluent from the first channel collected from port C using a selected gradient or concentration of mobile phase, for example, acetonitrile and water. The collected eluent can be fractionated and analyzed using a variety of known methods. In an embodiment, the eluent can be analyzed for glycans using MALDI-MS.

In accordance with an embodiment, the GIG-chip-LC apparatus of the present invention can be modified to include other separation or derivitization methods. For example, in an embodiment, the apparatus of the present invention can include one or more other channels packed with other stationary phases or supports which communicate with each other channel on the chip. In order to perform glycan permethylation, the apparatus can be created such that the second channel is connected to a third sodium hydroxide-packed channel either in the same substrate or chip, or on an adjacent substrate or chip. The extracted glycans from the GIG portion of the apparatus can be infused into the third sodium hydroxide-packed microchip for glycan permethylation, followed by separation using the chip-LC portion of the apparatus. Many other variations are contemplated.

In accordance with a further embodiment, the conjugation of the released glycans to the solid support is performed in the absence or presence of a catalyst. It will be understood by those of skill in the art that catalysts suitable for use with the methods of the present invention will include those compounds that can act as a Schiff-base intermediate in the reaction of the free reducing ends of the glycans with the hydrazide moieties on the solid support. In the inventive methods, the catalyst can be added to the mixture of the released glycans and solid support.

The solid substrate used to make the apparatus of the present invention may be any suitable material. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. In a preferred embodiment, the plastic used in the substrate is cyclic olefin polymer.

In accordance with an embodiment, the method of analyzing the eluted glycans includes, in certain embodiments, analyzing the glycans with a mass spectrometric method, an electrophoretic method, NMR, a chromatographic method or a combination thereof. In a further embodiment, the mass spectrometric method is LC-MS and LC-MS/MS using LC-Orbitrap, LC-FTMS, LC-LTQ, MALDI-MS including but not limited to MALDI-TOF, MALDI-TOF/TOF, MALDI-qTOF, and MALDI-QIT. Preferably, the mass spectrometric method is a quantitative MALDI-MS or LC-MS using optimized conditions. In still another embodiment, the electrophoretic method is CE-LIF. In yet another embodiment, methods such as capillary gel electrophoresis or capillary zone electrophoresis can be used with the inventive methods.

In other embodiments, the apparatus and methods of the present invention include quantifying the glycans using calibration curves of known glycan standards.

In yet another embodiment, the apparatus and methods of the present invention include methods for diagnostic or prognostic purposes.

In a further embodiment, the apparatus and methods of the present invention include methods for assessing the purity of the sample.

In some embodiments, the apparatus and methods used are methods of diagnosis and the pattern is associated with a diseased state. In one preferred embodiment, the pattern associated with a diseased state is a pattern associated with cancer, such as prostate cancer, melanoma, bladder cancer, breast cancer, lymphoma, ovarian cancer, lung cancer, colorectal cancer or head and neck cancer. In other preferred embodiments, the pattern associated with a diseased state is a pattern associated with an immunological disorder; a neurodegenerative disease, such as a transmissible spongiform encephalopathy, Alzheimer's disease or neuropathy; inflammation; rheumatoid arthritis; cystic fibrosis; or an infection, for example, a viral or bacterial infection. In other embodiments, the apparatus and methods used are methods of monitoring prognosis and the known pattern is associated with the prognosis of a disease. In yet another embodiment, the apparatus and methods used are for monitoring drug treatment and the known pattern is associated with the drug treatment. In particular, the apparatus and methods used are (e.g., analysis of glycome profiles) for the selection of population-oriented drug treatments and/or in prospective studies for selection of dosing, for activity monitoring and/or for determining efficacy endpoints.

Methods of analyzing glycans of glycoconjugates can also include cleaving the glycans from glycoconjugates using any chemical or enzymatic methods or combinations thereof that are known in the art. An example of a chemical method for cleaving glycans from glycoconjugates is hydrazinolysis or alkali borohydrate. Enyzmatic methods include methods that are specific to N- or O-linked sugars. These enzymatic methods include the use of Endoglycosidase H (Endo H), Endoglycosidase F (EndoF), N-Glycanase F (PNGaseF) or combinations thereof. In some preferred embodiments, PNGaseF is used when the release of N-glycans is desired. When PNGaseF is used for glycan release the proteins is, for example, first unfolded prior to the use of the enzyme. The unfolding of the protein can be accomplished with any of the denaturing agents provided above.

After the release of the glycan from the protein core, or when the glycans were already in free form (not part of a glycoconjugate), the sample can be purified, for instance, by precipitating the proteins with ethanol and removing the supernatant containing the glycans. Other experimental methods for removing the proteins, detergent (from a denaturing step) and salts include any methods known in the art. These methods include dialysis, chromatographic methods, etc. In one example, the purification is accomplished with a porous graphite column. In some preferred embodiments, everything but the glycans is removed from the sample. Samples can also be purified with commercially available resins and cartridges for clean-up after chemical cleavage or enzymatic digestion used to separate glycans from protein. Such resins and cartridges include ion exchange resins and purification columns, such as GlycoClean H, S, and R cartridges. Preferably, in some embodiments GlycoClean H is used for purification.

Purification can also include the removal of high abundance proteins, such as the removal of albumin and/or antibodies, from a sample containing glycans. In some methods the purification can also include the removal of unglycosylated molecules, such as unglycosylated proteins. Removal of high abundance proteins can be a desirable step for some methods, such as some high-throughput methods described elsewhere herein. In some embodiments of the methods provided, abundant proteins, such as albumin or antibodies, can be removed from the samples prior to the final composition analysis.

In other embodiments, the glycans can be modified to improve ionization of the glycans, particularly when MALDI-MS is used for analysis. Such modifications include permethylation. Another method to increase glycan ionization is to conjugate the glycan to a hydrophobic chemical (such as AA, AB labeling) for MS or liquid chromatographic detection. Examples of the methods are described further in the Examples below. In other embodiments, spot methods can be employed to improve signal intensity.

Any analytic method for analyzing the glycans so as to characterize them can be performed on any sample of glycans, such analytic methods include those described herein. As used herein, to “characterize” a glycan or other molecule means to obtain data that can be used to determine its identity, structure, composition or quantity. When the term is used in reference to a glycoconjugate, it can also include determining the glycosylation sites, the glycosylation site occupancy, the identity, structure, composition or quantity of the glycan and/or non-saccharide moiety of the glycoconjugate as well as the identity and quantity of the specific glycoform. These methods include, for example, mass spectrometry, NMR (e.g., 2D-NMR), electrophoresis and chromatographic methods. Examples of mass spectrometric methods include FAB-MS, LC-MS, LC-MS/MS, MALDI-MS, MALDI-MS/MS, etc. NMR methods can include, for example, COSY, TOCSY, NOESY. Electrophoresis can include, for example, CE-LIF, CGE, CZE, COSY, TOCSY, NOESY. Electrophoresis can include, for example, CE-LIF.

In an embodiment, the library consists of free or labeled glycoconjugates and fragments of the glycoconjugates, the fragments being the non-saccharide portions of the glycoconjugates. In one example, a library is generated from a sample, by isolating the glycoconjugates or free glycans or by cleaving the backbone of the glycoconjugates in the sample. The glycans or glycoconjugates can then be removed from the sample. The libraries so produced can be analyzed with the apparatus and methods provided herein. The libraries can also be used as a standard once characterized and methods of using such libraries are also provided.

In one embodiment, the inventive methods include a method of analyzing a sample with glycoconjugates includes isolating free forms of glycans or glycoconjugate, or cleaving the glycoconjugates by enzymatically or chemically removing the glycans from the glycoconjugates and mixing the sample with a standard. The sample mixed with the standard can then be analyzed. In one embodiment, the amounts of the glycoconjugates and non-saccharide moieties of the sample and standard are compared. In one aspect of the invention the standards are also provided.

Prior to analysis of the sample, the sample can also be degraded with a chemical or enzymatic method to cleave the glycans from any glycoconjugates in the sample. Examples of enzymatic methods are provided above and include, for example, the use of PNGase F, endoglycosydase H and endoglycosydase F or combinations thereof. Chemical methods have also been described above and include hydrazinolisis, alkali borohydrate or beta-elimination.

After chemical or enzymatic degradation the sample can then be performed in some embodiments. Purification methods were also provided above. Examples of particular purification methods include using solid phase extraction cartridges, such as graphitized carbon columns and C-18 columns.

As stated above, the glycosylation of a protein may be indicative of a normal or a disease state. Therefore, the apparatus and methods of the present invention are provided for diagnostic purposes based on the analysis of the glycosylation of a protein or set of proteins, such as the total glycome. The apparatus and methods provided herein can be used for the diagnosis of any disease or condition that is caused or results in changes in a particular protein glycosylation or pattern of glycosylation. These patterns can then be compared to “normal” and/or “diseased” patterns to develop a diagnosis, and treatment for a subject. For example, the apparatus and methods provided can be used in the diagnosis of cancer, inflammatory disease, benign prostatic hyperplasia (BPH), etc.

The diagnosis can be carried out in a person with or thought to have a disease or condition. The diagnosis can also be carried out in a person thought to be at risk for a disease or condition. “A person at risk” is one that has either a genetic predisposition to have the disease or condition or is one that has been exposed to a factor that could increase his/her risk of developing the disease or condition.

Detection of cancers at an early stage is crucial for its efficient treatment. Despite advances in diagnostic technologies, many cases of cancer are not diagnosed and treated until the malignant cells have invaded the surrounding tissue or metastasized throughout the body. Although current diagnostic approaches have significantly contributed to the detection of cancer, they still present problems in sensitivity and specificity.

In accordance with one or more embodiments of the present invention, it will be understood that the types of cancer diagnosis which may be made, using the apparatus and methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive apparatus and methods can provide any amount of any level of diagnosis, staging, screening, or other patient management, including treatment or prevention of cancer in a mammal

In accordance with an embodiment, the present invention provides a use of a glycan profile prepared using the apparatus and methods disclosed herein to diagnose a disease or condition in a subject, comprising comparing the glycan profile from a subject to a glycan profile from a normal sample, or diseased sample, and determining whether the sample of the subject has the disease or condition.

In accordance with the inventive apparatus and methods, the terms “cancers” or “tumors” also include but are not limited to adrenal gland cancer, biliary tract cancer; bladder cancer, brain cancer; breast cancer; cervical cancer; choriocarcinoma; colon cancer; endometrial cancer; esophageal cancer; extrahepatic bile duct cancer; gastric cancer; head and neck cancer; intraepithelial neoplasms; kidney cancer; leukemia; lymphomas; liver cancer; lung cancer (e.g. small cell and non-small cell); melanoma; multiple myeloma; neuroblastomas; oral cancer; ovarian cancer; pancreas cancer; prostate cancer; rectal cancer; sarcomas; skin cancer; small intestine cancer; testicular cancer; thyroid cancer; uterine cancer; urethral cancer and renal cancer, as well as other carcinomas and sarcomas.

### EXAMPLES

### SPGE Materials and Methods

Sialylglycopeptide (SGP) was provided by Dr. L-X Wang's lab from University of Maryland, School of Medicine. Tris(2-carboxythyl)phosphine (TCEP) and AminoLink resin were from Pierce (Thermo Scientific); peptide-N-glycosidase F (PNGase F), denaturing buffer and neuraminidase were from New England BioLabs; ribonuclease B (RNase B) from bovine pancreas, 2,5-dihydroxybenzoic acid (DHB), and N,N-dimethylaniline (DMA) were purchased from Sigma-Aldrich; μ-Focus MALDI plate and its holder were form Hudson Surface Technology; AXIMA Resonance—MALDI QIT/TOF mass spectrometry was from Shimadzu Biotech; human prostate cancer cell lines (22RV1, LnCap, PC3, and DU145) and cell culture media were purchased from American Type Culture Collection (ATCC); RIPA buffer was from Millipore; and protease inhibitor cocktail was from Roche. Serum was collected from healthy men with the approval of the Institutional Review Board of Johns Hopkins University and pooled for use. All other chemicals were purchased from Sigma unless specified.

Glycan extraction from peptides or proteins using SPGE. Conjugation: SGP glycopeptide or peptides from digested RNase B protein were conjugated to beads using reductive amination. Briefly, 50 μL of AminoLink resin (100 μl of 50% slurry) was incubated with peptides in 400 μL pH 10.0 buffer (40 mM sodium citrate and 20 mM sodium carbonate) at room temperature for 6 hours with mixing. After rinsing the resin with 400 μL of 50 mM phosphate buffer (pH 7.4) twice, peptides on AminoLink resin were further reduced by addition of 50 mM NaCNBH₃ in 50 mM phosphate buffer (pH 7.4) at room temperature for 4 hours with mixing. After incubation, the beads were washed with 1 M Tris-HCl (400 μL, pH 7.6) twice before addition of 400 μL of 1 M Tris-HCl (pH 7.6), 50 mM NaCNBH₃ to block the unreacted aldehyde sites on the bead surface for 30 minutes. To conjugate serum proteins to beads, proteins from 20 μg were first denatured in 100 μL of a solution consisting of 10 μL 10× denaturing buffer (New England Biolabs) and 90 μL pH 10.0 buffer for 10 minutes at 100° C. before conjugation to AminoLink resin. The proteins or peptides immobilized on beads were washed three times with 400 μL of 1 M NaCl, three times with H₂O, and three times with 5 mM NH₄HCO₃.

Glycan release: For additional exoglycosidases treatment, 0.5 μL of neuraminidase was added into the beads conjugated with SGP glycopeptide with 10 μL of 50 mM sodium citrate (pH 6.0) and incubated at 37° C. for 2 hours to remove sialic acid groups. The enzyme and chemical reagent were removed by washing with 400 μL of 1 M NaCl, then H₂O, and then 5 mM NH₄HCO₃. Finally, 1 μL of PNGase F with 39 μL of 5 mM NH₄HCO₃ was added to the bead mixture and incubated at 37° C. for 2 hours to release N-glycans. The supernatant was collected and vacuum dried. Methanol was used to stabilize sialic acid groups on glycans.

Analysis of glycans using MALDI-MS. Glycans extracted from SGP, RNase B, serum, or cells using SPGE were analyzed by AXIMA MALDI Resonance mass spectrometer (Shimadzu). We used 4 μL DMA in 200 μL DHB (100 μg/μL in 50% acetonitrile, 0.1 mM NaCl) as matrix to increase the detection of sialylated glycans. The DHB-DMA spots formed uniform crystals and increased sialylated glycan stability by increasing laser power absorption and ionization efficiency. The laser power was set to 100 for 2 shots each in 50 locations per spot. The average MS1 spectrum was used for glycan assignment by comparison to a database of N-glycans previously analyzed by MALDI-MS/MS in our lab. MS/MS spectra were used to confirm the assigned glycans.

Glycan Analysis of Proteins from Cell Lines Using SPGE and Lectin Blot.

For glycan capture from cancer cells using SPGE, cells in a 10-cm dish at subconfluence were rinsed with 500 μL of 1×PBS buffer (50 mM) 5 times and harvested in 500 μL of pH 10.0 buffer (40 mM sodium citrate, 20 mM sodium carbonate, pH 10.0). The cells were sonicated for 3 minutes at 30-second intervals with cooling on ice between sonications. The dissolved proteins were brought to a final volume of 1000 μL with pH 10.0 buffer. The concentrations of protein samples were measured using a BCA assay kit (Pierce). For each cell line, an equal amount of proteins (4 μg) was used for protein conjugation following the SPGE procedure described above.

The lectin blot method was described previously. Briefly, cells in a 10-cm dish at subconfluence were lysed in 1×RIPA buffer and incubated on ice for 10 minutes. After incubation, samples were centrifuged at 15,000×g for 15 minutes. Protein concentration was determined using BCA protein assay kit (Pierce). For each sample, an equal amount of proteins (10 μg) was run on 4-12% NuPAGE gel (Invitrogen) and then transferred to nitrocellulose membrane (Invitrogen). Gels were stained by Coomassie Brilliant Blue (CBB) to determine the amount of protein loading. Membranes were blocked with 1× non-carbohydrate blocking buffer (Vector Laboratories) overnight at 4° C. and then incubated with 0.5 μg/ml biotinylated Aleuria Aurantia Lectin (AAL) and biotinylated Concanavalin A (Con A) (Vector Laboratory) in TBST for 1 hour at room temperature. After washing with TBST three times, lectin-reactive proteins were detected using a Vectastain ABC kit (Vector Laboratory) and an ECL kit (Invitrogen).

Materials and Methods for Sialylated Glycan Analysis.

Bovine fetuin, human serum, p-toluidine, aniline, dimethylaniline (DMA), 2,5-dihydroxybenzoic acid (DHB), and EDC were purchased from Sigma Aldrich (St Louis Mo.) p-toluidine-d9 was purchased from CDN isotopes (Pointe-Claire, Quebec Canada). Acetohydrazide was purchased from Tokyo Chemical Industry Co. Ltd (Tokyo, Japan). PNGase F was obtained from New England Biolabs (Ipswich, Mass.) Amino link coupling resin was purchased from Pierce (Thermo Fisher Scientific Inc.; Rockford, Ill.).

Protein Binding to Solid Support.

Proteins were immobilized to amino link beads via reductive amination. Briefly, AminoLink resin (200 μL) was loaded onto snap-cap spin-column, centrifuged at 2000 g for 1 minute. Resin was washed with 450 μL of pH 10 buffer (Sodium citrate 100 mM and sodium carbonate 50 mM) followed by centrifugation. The washing step was repeated twice. Proteins dissolved in pH 10 buffer were loaded onto prepared AminoLink resin in snap-cap spin-column in 1 mg/200 μL beads ratio. The volume was adjusted to 450 μL using pH 10 buffer. Sample-resin mixture was incubated at room temperature overnight. The mixture was centrifuged at 2000 g to remove any unbound protein. Resin was rinsed by 1×PBS buffer (Sigma-Aldrich; pH 7.4; 450 μL) three times. PBS buffer in the presence of 50 mM sodium cyanoborohydride (450 μL) was added to resin (spin-column capped during each incubation step). After a four hour incubation, supernatant was removed via centrifugation (2000 g) and 450 μL of 1 M Tris-HCl (pH 7.6) in the presence of 50 mM sodium cyanoborohydride was added to block un-reacted aldehyde sites of resin. The blocking process was terminated after 1 hour, followed by washing of resin with PBS twice, 1M NaCl 1.5M twice and water three times.

Derivatization of Sialic Acids on Proteins Conjugated to Solid Support and Release of N-glycans.

Proteins conjugated on solid support were mixed with 450 μL of 1M p-toluidine and 40 μL of EDC at pH 4.5 adjusted using HCl. After incubation for 4 hours, beads were washed twice with 500 μL of 1M NaCl and water followed by 50 mM ammonium bicarbonate, three times for each solution, and treated with 100 units of PNGase F in 0.3 mL of 50 mM ammonium bicarbonate overnight at 37° C. to release N-glycans from proteins. Nglycans released in the supernatant were subsequently collected. The N-glycans released from glycoproteins were further purified and concentrated over Carbograph columns (Extract-Clean SPE Carbo 150 mg; Grace Division Discovery Science; Deerfield, Ill.) and eluted in 0.1% TFA 50% acetonitrile/water following the manufacturer's instruction (Grace Davison Discovery Sciences, Milwaukee, Wis.). Glycans were then dried in Savant Speed-Vac (Thermo Scientific, Asheville, N.C.).

Mass Spectrometric Analysis of Sialylated Glycans.

Glycans were resuspended in 20 μL of water. Sample 1.5 μL was mixed 1.5 μL of matrix on a 384-well goals MALDI plate (Hudson Surface Technology, Fort Lee, N.J.). DHB matrix solution was prepared by dissolving 100 mg of DHB in 1 mL of a 1:1 solution of water and ACN followed by addition of 40 μL of dimethylaniline. Glycans were analyzed by Shimadzu AXIMA Resonance Mass Spectrometer (Shimadzu, Columbia, Md.) in positive mode.

Materials for GIG-chip-LC. AminoLink resin was from Pierce (Thermo Fisher Scientific Inc.; Rockford, Ill.). Peptide-N-glycosidase F (PNGase F), denaturing buffer and G7 were from New England BioLabs (Ipswich, Mass.). p-toluidine, ribonuclease B (RNase B) from bovine pancreas, 2,5-dihydroxybenzoic acid (DHB), N,N-dimethylaniline (DMA), and carbon (mesoporous; particle size, 45±5 μm; pore size, 100±10 Å), were purchased from Sigma-Aldrich (St. Louis, Mo.). Mouse heart and serum were provided by Dr. Xingde Li from Biomedical Engineering Department of Johns Hopkins University. Pooled human sera were collected from healthy men with the approval of the Institutional Review Board of Johns Hopkins University. All other chemicals were purchased from Sigma unless specified.

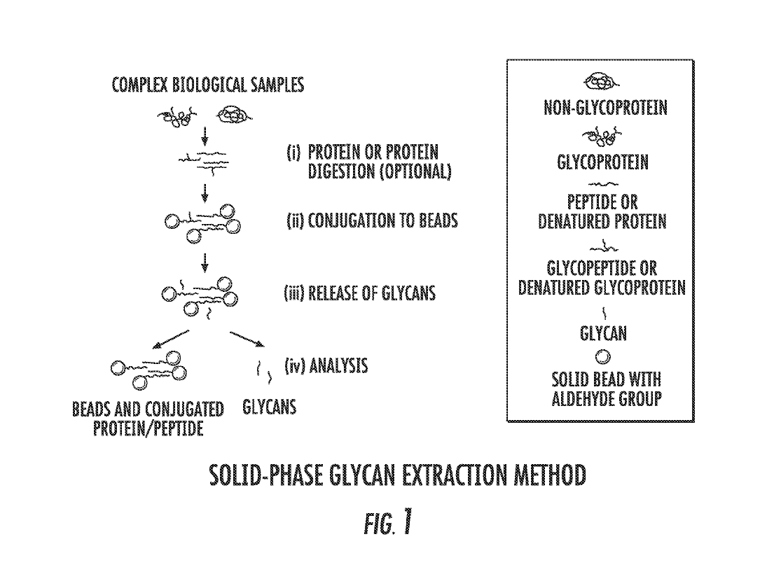

Microchip Fabrication. The microfluidic devices of the present invention were fabricated from cyclic olefin polymer (COP) (Zeonor 1020R; 2 mm×50 mm×100 mm; Zeon Chemicals L.P.; Louisville, Ky.). Three reservoirs (8) (A, B and C, FIG. 1B) were drilled into substrate for needle insertion using a drilling end mill (650 μm diameter) by an MDX-650A computer numerical control (CNC) system (Roland ASD; Lake Forest, Calif.) (Lab Chip 2009, 9, 50-55) (FIG. 1B). Each needle was connected to the external components i.e. syringe pump, syringe needle, and elution collector through a capillary (ID: 100 μm, OD: 360 μm; Polymicro Technologies; Phoenix, Ariz.). A second end mill (150 μm diameter) was used for micromachining the constrained channel portions of the first (3) and second (6) channel at the depth of 50 μm (FIG. 1A). A third end mill (800 μm diameter) was used to fabricate the remainder of the first (2) and second (4) channels at the depth of 800 μm (FIG. 1A). The machined chip was sonicated in DI water for 30 minutes to ensure removal of debris along channel edge.

COP substrates with microchannel features and cover sheets containing reservoirs were cleaned by sequential washing with methanol, isopropanol, and distilled water, and then dried with nitrogen gas. Both COP sheets were degassed overnight in a vacuum oven at 75° C. The cover sheet was fixed on a glass plate and incubated in a 4 L glass flask (flat flange and bottom) containing cyclohexane such that the level of liquid cyclohexane was 5 cm under COP surface. The COP was incubated for 8 minutes to adsorb cyclohexane vapor. Both COP sheets were placed in immediate contact to form an initial solvent bond, with trapped air bubbles removed manually. The temporarily bonded COP sheets were placed between two glass plates, with teflon films between both COP and glass interfaces to reduce shear forces during bonding. The final bonding was performed using a hot press (Auto Four; Carver Inc.; Wabash, Ind.) at 625 lbs/in² (45 kg/cm²), 30° C. for 2 minutes. The bonded chip was allowed to dry in a vacuum oven at 75° C. for at least 3 hours before insertion of the stainless steel needles (25 mm, 22 gauge surgical needle, 710 μm O.D., 400 μm I.D.; Hamilton, Reno, Nev., USA). The microchip with inserted needles was then annealed in a vacuum oven at 75° C. for 4 hours. Further fabrication details can be found in the literature (Anal. Chem. 2009, 81, 2545-2554).

The nominal channel geometry for the first and second channels was 800 μm×800 μm and each of the channel segments were designed with narrowed channel cross-section, identified as a “constrained portion” shown as (3, 6) in FIG. 17A. These constrained channel portions function as a virtual valve to prevent beads/particles movement and loss during GIG and chip-LC processes. The second channel (4) from port B to interface (5) was packed with AminoLink coupling beads. The first channel (2) from port A to C was filled with porous graphitized carbon particles.

### Example 1

### Solid-Phase Glycan Extraction and Modification of Immobilized Glycans (SPGE)

In accordance with one or more embodiments, the methods of the present invention include the following steps (FIG. 1): i) Protein/peptide conjugation: The proteins or peptides were coupled to aldehyde groups of a solid support through reductive amination of N-terminal and/or lysine residues of proteins or peptides. After coupling, unreacted aldehydes on beads were blocked using Tris buffer via the same reductive amination reaction and unconjugated proteins and other contaminants were washed away. ii) Glycan release: The reaction buffer was exchanged and glycans were released. iii) Mass spectrometry analysis: The collected glycans were dried and spotted onto a MALDI target without additional sample clean-up. Overall, the SPGE method provides a rapid and robust analysis platform for glycan profiling in complex biological samples.

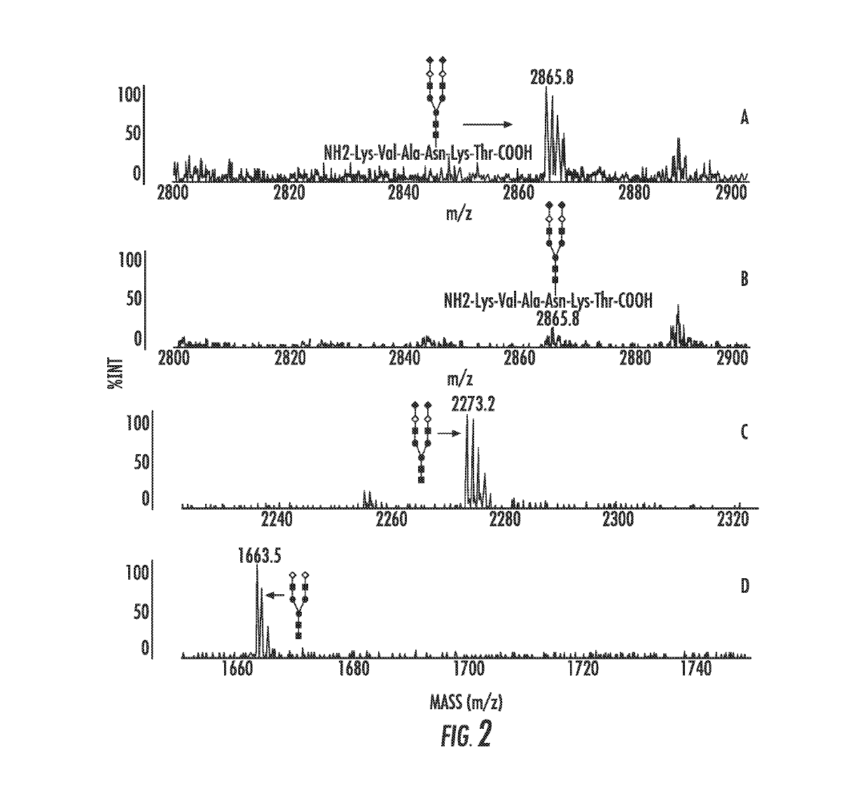

To validate the methods of the present invention, we first used a sialylglycopeptide (SGP) with known N-glycan structure. The SGP has a biantennary N-glycan linked to Asn residue of a peptide containing six amino acids (Lys-Val-Ala-Asn-Lys-Thr, FIG. 2A). The SGP was treated as described above and analyzed after each step. FIG. 2A shows detection of the SGP in solution before conjugation ([M+H]+ ion with mass of 2865.8 Da. After 6-hour conjugation to beads, little un-conjugated SGP remained in solution indicating efficient conjugation of the SGP to beads (FIG. 2B). After washing the beads to remove the unconjugated molecules, PNGase F was used to release N-glycan from SGP in 5 mM NH₄HCO₃ at 37° C. After PNGase F treatment, biantennary N-glycan with sialic acids ([M+2CH₂+Na]⁺ ion with mass of 2273.2 Da) was observed by MALDI-MS (FIG. 2C). A two-hour incubation resulted in maximum recovery of the glycan. This result showed that the SPGE method can be used to capture glycans from glycoproteins. Moreover, the SPGE method provides a solid-phase platform for glycan modification/derivatization. After SGP was coupled to beads, the conjugated SGP was treated with neuraminidase to remove the sialic acid from SGP before the glycan was released from glycopeptides immobilized on beads. After neuraminidase treatment, an intense signal for the [M+Na]+ ion at 1663.5 Da, corresponding to the de-sialylated glycan, was observed (FIG. 2D).

### Example 2

### Performance of Glycan Analysis Using SPGE

To investigate the sensitivity of solid-phase SPGE for glycan analysis, 0, 0.01, 0.05, 0.1, 0.5, and 1 μg of SGP were used in conjugation and release experiments, and the isolated glycan was analyzed by MS in triplicate. The limit of detection of the glycan was that obtained from 0.1 μg of SGP peptide; the S/N ratio was greater than 20 for the sialylated biantennary N-glycan peak at 2273.2 Da.

To determine whether the isolation of glycan using SPGE was quantitative, peak areas in the MS spectra of sialylated biantennary N-glycan (m/z=2273.2) isolated from 1 μg of SGP in triplicate were calculated. The peak areas were normalized to an internal standard (angiotensin). The CV of glycan analyses from triplicate isolations was 12.87%, which indicates that SPGE can be used for quantitative analysis of glycans.

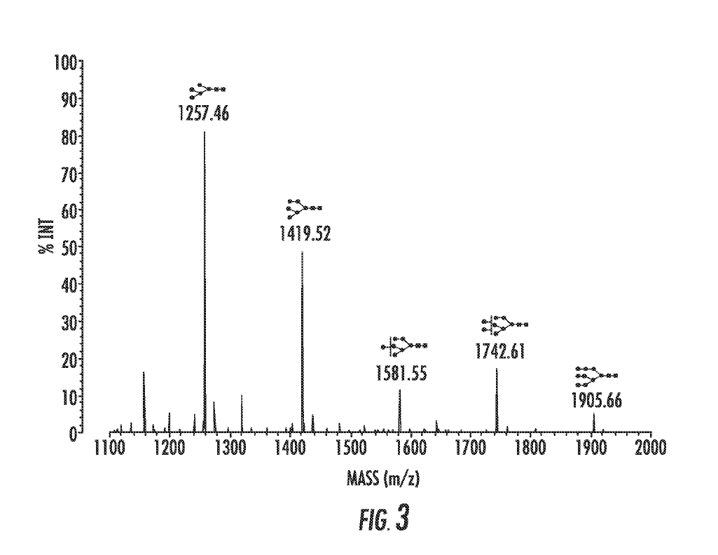

To assess the potential of the SPGE method for glycan profiling from complex samples, we first determined specificity of glycan extraction from a mixture of tryptic peptides from a glycoprotein. RNase B is a glycoprotein with five N-linked high-mannose structures 15, Man-5, Man-6, Man-7, Man-8, and Man-9. All five of these previously reported glycans with high-mannose structures were detected using the SPGE procedure (FIG. 3). These results indicate that SPGE can be used to specifically recover most glycans from a mixture of peptides of a glycoprotein as determined from intensity and number of glycans identified.

### Example 3

### Glycan Extraction from Complex Serum Samples Using SPGE

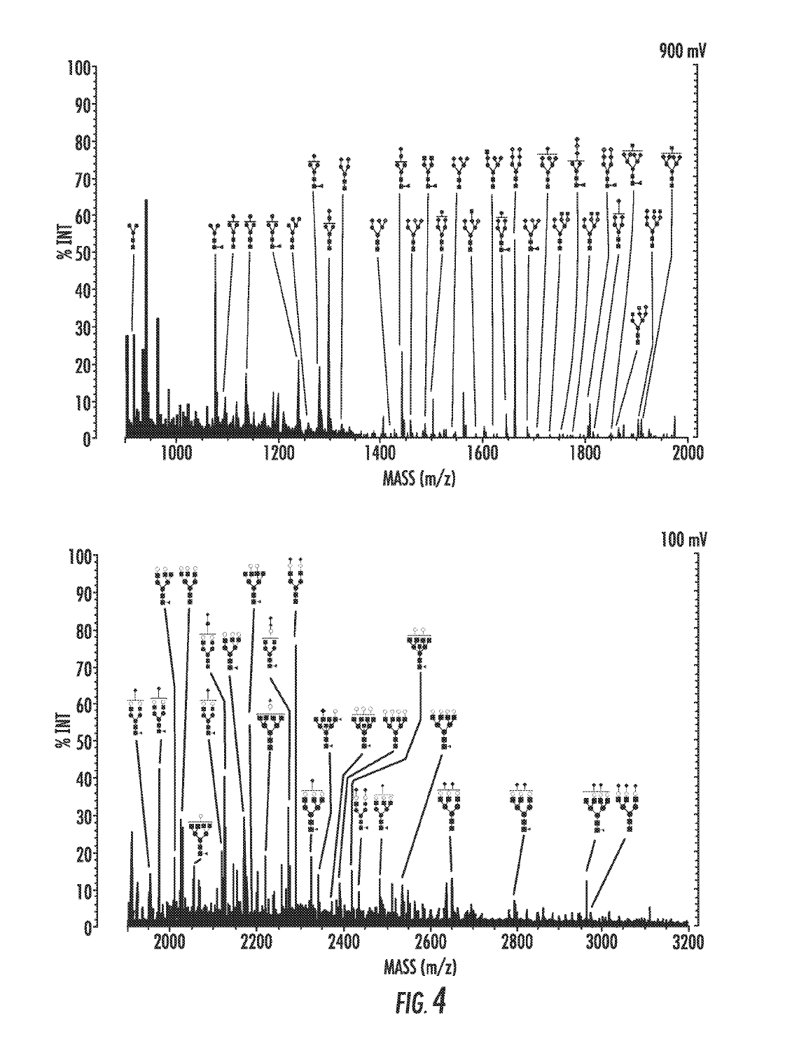

We then applied the SPGE method to the analysis of glycans from a very complex sample—human serum. After coupling of 20 μg of serum proteins to solid support, the beads were washed to remove unconjugated proteins and other molecules present in the sample. The glycans were released from the bead-bound glycoproteins by treating the beads with PNGase F. The glycan solution was dried and dissolved in 40 μL water. Of this, 2 μL (the equivalent of 1 μg of serum proteins) was analyzed by MALDI-MS and MS/MS. Without additional sample clean up or further glycan separation, we were able to identify 60 N-glycans (FIG. 4). Fifty-one glycans were verified by MS/MS analyses of the serum sample (data not shown).

TABLE 1
The identified glycans from serum using SPGE and MS.

| Glycan | MW (Da) | [M + nNa]⁺ | n |
|---|---|---|---|
| 2Hex:1HexNAc:1Hex* | 749.01 | 771.99 | 1 |
| 4Hex:1HexNAc | 851.61 | 874.59 | 1 |
| 3Hex:2HexNAc | 892.56 | 915.54 | 1 |
| 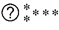 | 910.32 | 933.30 | 1 |
| 2Hex:2HexNAc:1Hex* | 952.48 | 975.46 | 1 |
| 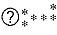 | 1056.29 | 1079.27 | 1 |
| 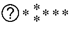 | 1072.37 | 1095.35 | 1 |
| 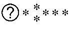 | 1113.40 | 1136.38 | 1 |
| 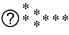 | 1234.42 | 1257.40 | 1 |
| 4Hex:3HexNAc | 1256.92 | 1279.90 | 1 |
| 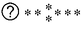 | 1275.45 | 1298.43 | 1 |
| 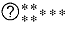 | 1316.48 | 1339.46 | 1 |
| 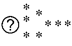 | 1396.48 | 1419.46 | 1 |
| 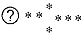 | 1421.51 | 1444.49 | 1 |
| 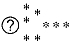 | 1437.50 | 1460.48 | 1 |
| 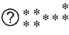 | 1462.53 | 1485.51 | 1 |
| 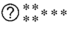 | 1478.53 | 1501.51 | 1 |
| 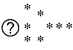 | 1519.56 | 1542.54 | 1 |
| 3Hex:HexNAc:2Hex* | 1538.35 | 1561.33 | 1 |
| 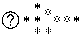 | 1558.53 | 1581.51 | 1 |
| 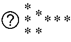 | 1599.55 | 1622.53 | 1 |
| 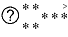 | 1624.59 | 1647.57 | 1 |
| 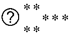 | 1640.58 | 1663.56 | 1 |
| 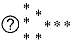 | 1665.61 | 1688.59 | 1 |
| 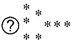 | 1681.61 | 1704.59 | 1 |
| 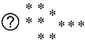 | 1720.58 | 1743.56 | 1 |
| 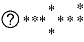 | 1710.92 | 1756.88 | 2 |
| 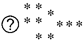 | 1761.61 | 1784.59 | 1 |
| 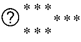 | 1786.64 | 1809.62 | 1 |
| 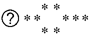 | 1767.81 | 1813.77 | 2 |
| 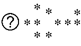 | 1827.67 | 1850.65 | 1 |
| 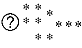 | 1843.66 | 1866.64 | 1 |
| 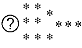 | 1882.64 | 1905.62 | 1 |
| 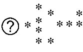 | 1886.63 | 1909.61 | 1 |
| 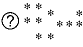 | 1907.67 | 1930.65 | 1 |
| 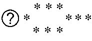 | 1929.11 | 1975.07 | 2 |
| 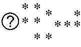 | 1989.72 | 2012.70 | 1 |
| 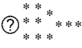 | 2005.71 | 2028.69 | 1 |
| 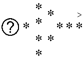 | 2031.99 | 2054.97 | 1 |
| 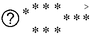 | 2074.81 | 2120.77 | 2 |
| 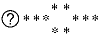 | 2057.78 | 2126.72 | 3 |
| 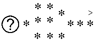 | 2162.87 | 2172.06 | 1 |
| 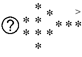 | 2162.87 | 2185.85 | 1 |
| 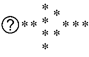 | 2176.64 | 2222.60 | 2 |
| 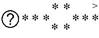 | 2200.47 | 2269.41 | 3 |
| 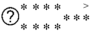 | 2219.94 | 2288.38 | 3 |
| 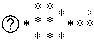 | 2294.34 | 2317.32 | 1 |
| 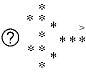 | 2320.38 | 2343.36 | 1 |
| 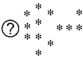 | 2352.25 | 2375.23 | 1 |
| 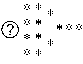 | 2368.29 | 2391.27 | 1 |
| 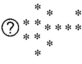 | 2396.25 | 2419.23 | 1 |
| 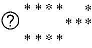 | 2364.16 | 2433.10 | 3 |
| 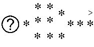 | 2439.05 | 2485.01 | 2 |
| 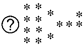 | 2514.95 | 2537.93 | 1 |
| 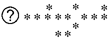 | 2504.82 | 2550.78 | 2 |
| 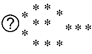 | 2582.63 | 2651.57 | 3 |
| 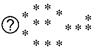 | 2729.33 | 2798.27 | 3 |
| 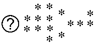 | 2890.86 | 2959.80 | 3 |
| 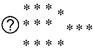 | 2872.90 | 2964.82 | 4 |
| 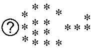 | 3656.06 | 3747.98 | 4 |

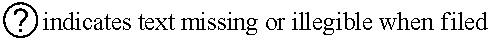

### Example 4

### Fucosylation and Mannosylation of Cancer Cells

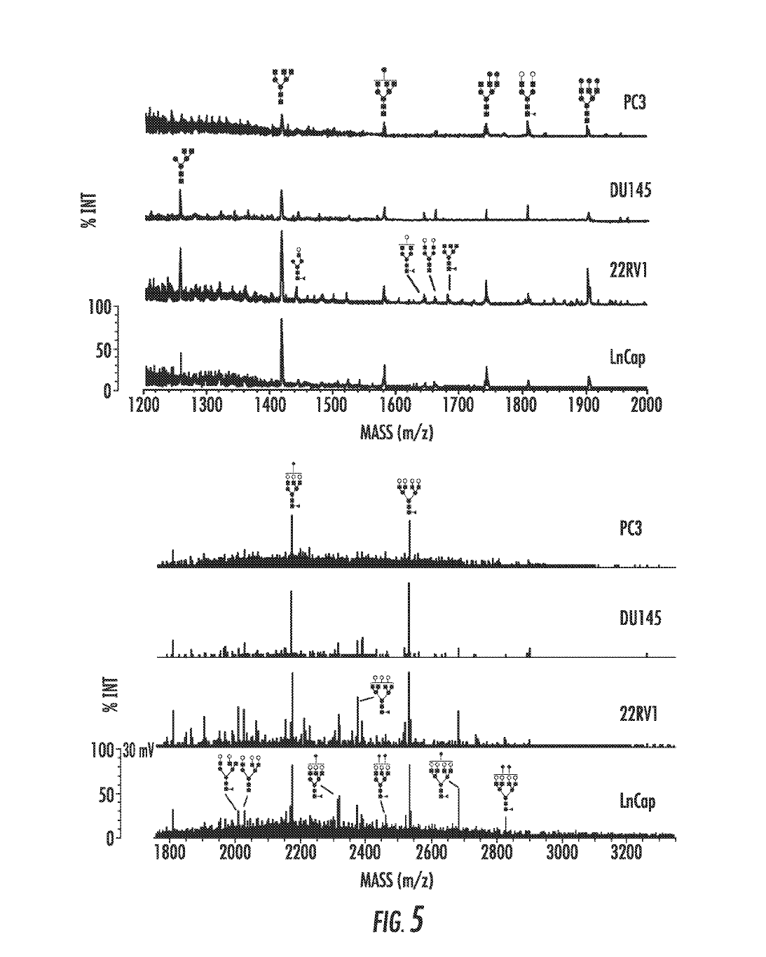

Glycosylation changes such as fucosylation and mannosylation are associated with cancer progression. To determine whether the solid-phase glycan extraction method could be used for the profiling of fucosylated and mannosylated glycans from cancer cells with different phenotypes, we analyzed N-linked glycans from LnCap and 22RV1, androgen-dependent, less invasive cells, and from PC3 and DU145, androgen-dependent, invasive cells. Proteins from cell lysates (4 μg) were immobilized on beads, and glycans were desialylated using neuraminidase to reduce the complexity for targeted analyses of fucosylated and mannosylated N-glycans. The N-linked glycans were released and analyzed by mass spectrometry. In the MALDI-MS spectra, we observed three major peaks at 1809.4 Da, 2174.7 Da, and 2539.9 Da identified as core fucosylated bi-antennary, tri-antennary and quad-antennary glycans, respectively in all four cell lines (FIG. 5 top and bottom). We also observed that each core fucosylated glycan was further fucosylated on its antennary branches (FIG. 5 bottom).

To quantify relative abundance of fucosylated glycans in different cell lines, maltoheptaose (DP7) (1175.4 Da, mono-Na⁺) was added as internal standard. The intensities of fucosylated glycans from each of the four cell lines was semi-quantified by normalizing to the DP7 intensity in each analysis (Table 2). Tri-antennary and quad-antennary core fucosylated glycans were detected in all four cancer cell lines, but PC3 cells had lower levels of tri-antennary and quad-antennary core fucosylated glycans than the other three cell lines (Table 2). Fucosylation of one or two fucoses on tri- or quad-antennary branches were abundant in the less invasive LnCap and 22RV1 cells, whereas lower amounts of tri- or quad-antennary fucosylated glycans were detected in the more invasive PC3 and DU145 cells. In contract to tri-antennary and quad-antennary glycans, higher levels of bi-antennary glycans were detected in the more invasive PC3 and DU145 cells than in the LnCap and 22RV1 cells (FIG. 5 top and Table 2).

TABLE 2
Quantitative analysis of fucosylated glycans on four prostate cancer cell
lines.

| Cell | 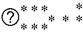 | 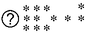 | 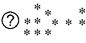 | 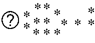 | 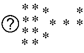 | 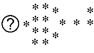 | 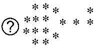 |
|---|---|---|---|---|---|---|---|
| LnCap | 0.172 | 0.927 | 0.546 | 0.252 | 1.023 | 0.615 | 0.389 |
| 22RV1 | 0.143 | 1.347 | 0.426 | 0.207 | 1.220 | 0.577 | 0.195 |
| DU145 | 0.296 | 1.104 | 0.245 | 0.051 | 1.155 | 0.158 | 0.040 |
| PC3 | 0.266 | 0.622 | 0.113 | 0.049 | 0.602 | 0.089 | 0.020 |

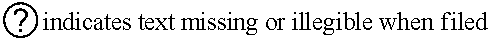

Five high-mannose glycans (Man5, Man6, Man7, Man8, and Man9) were detected in the four cancer cell lines in 1000 to 2000 Da range (FIG. 5 top). The high-mannose glycans were quantitatively analyzed using an approach similar to that used for analysis of fucosylated glycans (Table 3). Levels of high mannose glycans were higher in less invasive LnCap and 22RV1 cells than in move invasive DU145 and PC3 cells. However, higher level of large high-mannose glycans (Man9 and Man8) comparing to small high-mannose glycans (Man5 and Man6) was detected in more aggressive DU145 and PC3 cells, indicating that different species of high-mannose glycans characterize each cell line and less small high-mannose glycans were present in Man5/Man6 forms in DU145 and PC3 cell lines.

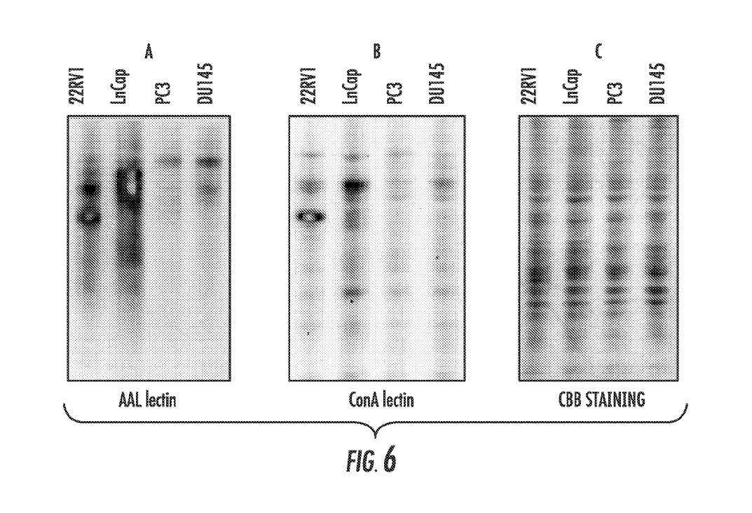

To determine whether altered fucosylation was detected in glycoproteins, cell lysates were analyzed by AAL lectin, which recognizes fucosylated glycoproteins. The AAL lectin detected different fucosylations on different prostate cancer cell lines (FIG. 6A). LnCap cells had the highest levels of fucosylation of the cell lines evaluated, and the fucosylation was apparently present on a number of glycoproteins since a wide range of molecular weights were detected. The fucosylation levels of 22RV1 cells were slightly lower than those of LnCap cells, but mainly on two particular glycoproteins. Both PC3 and DU145 contained lower levels of fucosylated glycoproteins, mainly on a single large glycoprotein. These data are consistent with the results from the analysis of fucosylated glycans, in which lower levels of fucosylated glycans were detected on PC3 and DU145 cells than on LnCap and 22RV1 cells.

ConA lectin was used to detect the high mannose glycoproteins from cell lysates. ConA staining showed high mannosylation on several glycoproteins from LnCap and 22RV1 cells (FIG. 6B) but low levels of mannosylated glycoproteins on PC3 and DU145 cells (FIG. 6B). Coomassie Brilliant Blue (CBB) staining indicated that the four cancer cells had similar protein constituents and protein concentrations (FIG. 6C). Thus, lectin analysis verified the glycan differences detected by the SPGE method in phenotypically different prostate cancer cell lines.

TABLE 3
Quantitative analysis of mannosylated glycans on four prostate cancer cell lines.

| | Intensity | | | | |
|---|---|---|---|---|---|
| Cell | 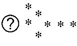 | 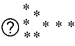 | 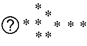 | 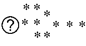 | 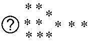 |
| LnCap | 0.319 | 0.670 | 0.266 | 0.255 | 0.136 |
| 22RV1 | 0.389 | 0.724 | 0.204 | 0.289 | 0.482 |
| DU145 | 0.154 | 0.263 | 0.118 | 0.146 | 0.057 |
| PC3 | 0.093 | 0.217 | 0.124 | 0.160 | 0.118 |

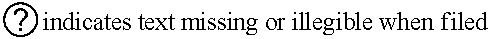

### Example 5

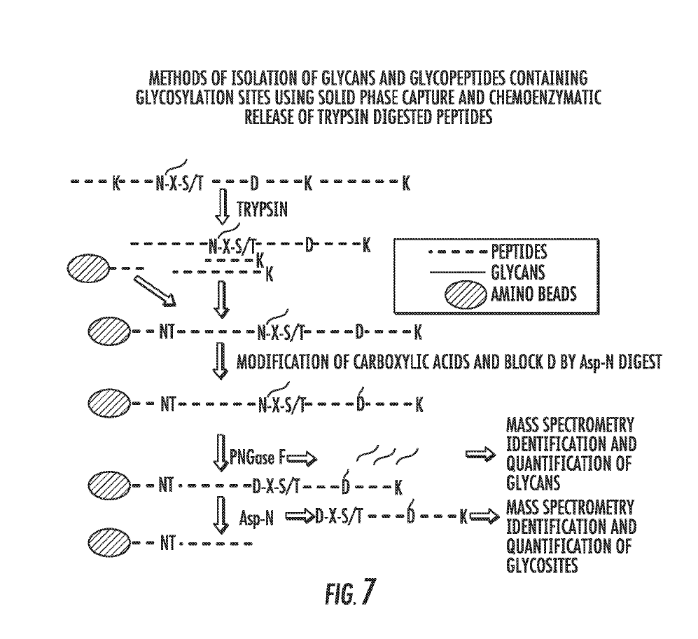

The method for solid-phase extraction of glycopeptides and glycans SPEGAG method was applied to the analysis of human serum with the following steps (FIG. 7):
1. 1 mg human serum was digested by trypsin overnight.
2. The sample was cleaned up with a C18 cartridge using standard methods.
3. Guanidylation (Translate Lysine to Homoarginine), the sample is guanidinylated using standard protocol.
4. The sample was cleaned up with a C18 cartridge using standard methods.
5. The peptide mixture is then coupled to a solid support by reductive amination in PBS buffer, pH 7.4 with 50 mM NaCNBH₃.
6. Blocking unused aldehyde groups on the solid support.
7. Labeling the acid groups by aniline. (add internal standard peptides to check label efficiency). The labeling can be isotopic or isobaric tags to introduce mass difference for glycan and glycopeptide quantification.
8. An Asp-N digest of the sample is then performed to remove any unlabeled aspartic acid groups.
9. The samples is then digested with PNGase F to release N-glycans from the substrate.
10. Analyzing the released glycans by MS to identify and quantify glycans.
11. Digesting the peptides on the solid substrate with Asp-N to release N-glycopeptides at the N-terminal of the glycosylation motif (▾NXT/S).
12. Analyzing the released glycans by MS to identify and quantify glycans.
13. Search LC-MS/MS against database: for example, ipi.HUMAN.v3.87N_KN.fasta (all N-X-S/T were replaced by K-N-X-S/T so that I could select trypsin as enzyme) Enzyme Name: Trypsin (Full) Maximum Missed Cleavage Sites: 1.
   Using dynamic Modifications:Max. Modifications Per Peptide: 7

### C-Terminal Modification: AnilineCtermi/+75.047 Da (Any C-Terminus)

### 1. Dynamic Modification: Deamidated/+0.984 Da (N)

### 2. Dynamic Modification: Guanidinyl/+42.022 Da (K) Modify Lysine to HomoArginine

### 3. Dynamic Modification: Oxidation/+15.995 Da (M)

### 4. Dynamic Modification: AnilineDE/+75.047 Da (D, E)

### 5. Dynamic Modification: Guanidylaniline/+117.069 Da (K)

### 5. Static Modifications:

### 1. Static Modification: Carbamidomethyl/+57.021 Da (C)

### Target FDR (Relaxed): 0.05

There were 61 N-glycopeptides from 45 N-glycoproteins identified in human serum by SPEGAG method. The specificity is 66.30% at peptide level and 70.31% at protein level (92 peptides from 64 proteins were identified).

### Example 6

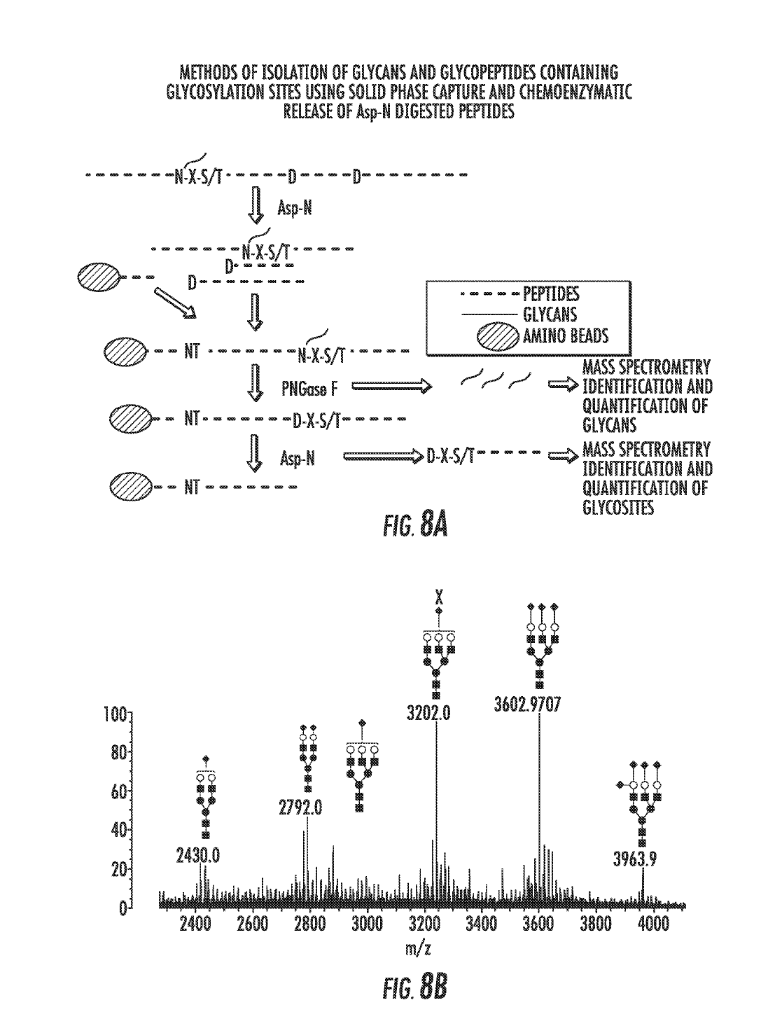

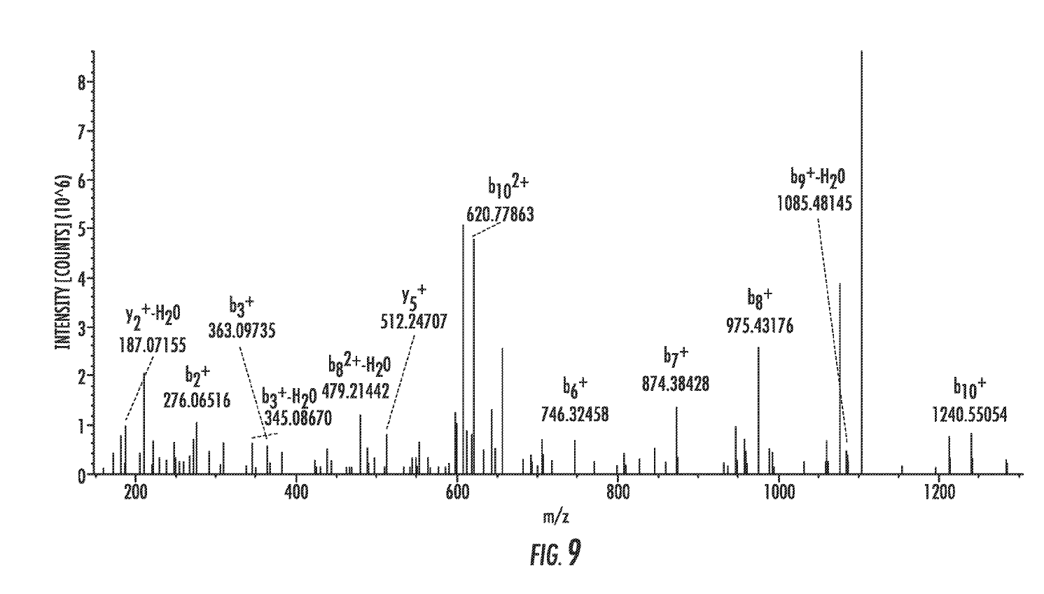

In FIG. 8A, we see an example of another embodiment of the SPEGAG method of the present invention. Using this strategy, both N-glycopeptides and N-glycans from fetuin can be isolated. Besides, 108 unique N-glycopeptide from 84 N-glycoproteins were identified from OVCAR-3 cell lines (FIG. 8B).

### Example 7

### Solid Phase Labeling of Sialylated Glycans

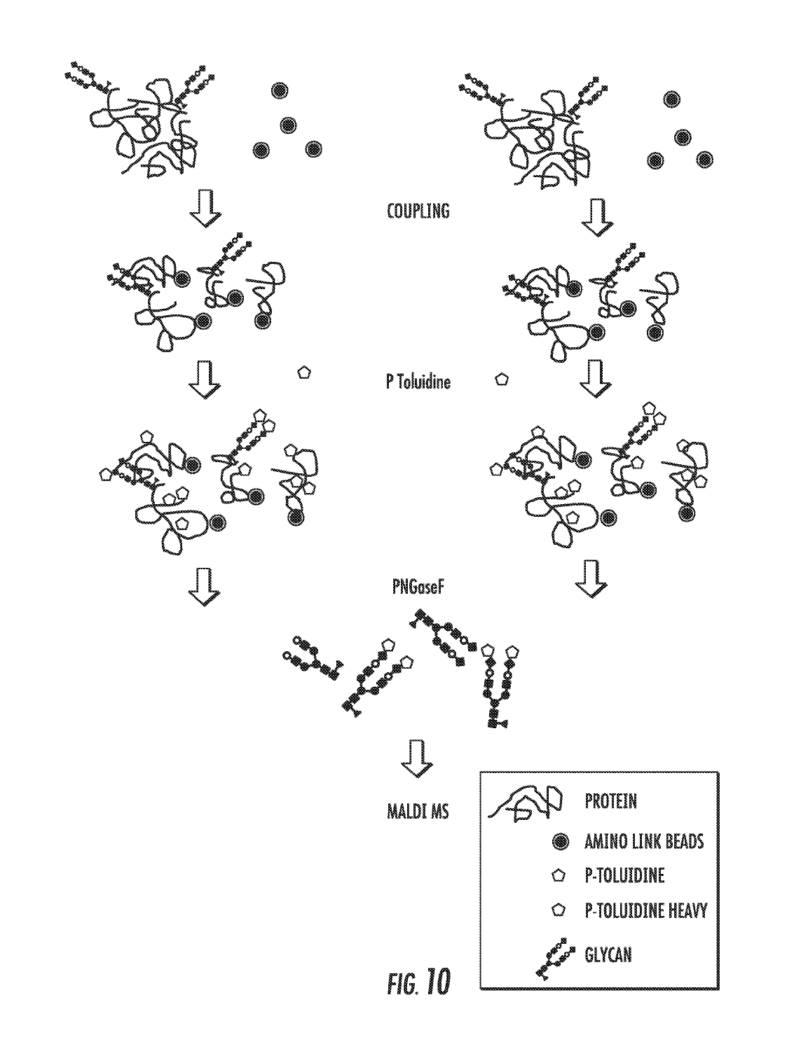

The derivatization of sialylated glycans was successfully performed on protein glycans conjugated to the solid phase. Labeled sialylated glycans were subsequently released from solid support for mass spectrometry analysis (FIG. 10). First, proteins were conjugated to solid support by reduction amination. Second, the carboxylic groups of sialic acids were modified by p-toluidine in the presence of EDC while glycans were still attached to the protein on solid support. Third, N-glycans were then released from proteins by PNGase F treatment. Fourth, the release glycans were analyzed using mass spectrometry in positive mode. When light and stable isotopic heavy p-toluidine reagents were used to derivatize sialic acids, seven mass unit differences between light and heavy tags for each sialic acid could be effectively resolved in the mass spectrum. The mass difference allowed the identification of the number of sialic acids in the glycan structures when 1:1 mix of light and heavy reagents were used for glycan labeling. The light and heavy reagents were also applied to different samples for the relative quantification of sialylated glycans.

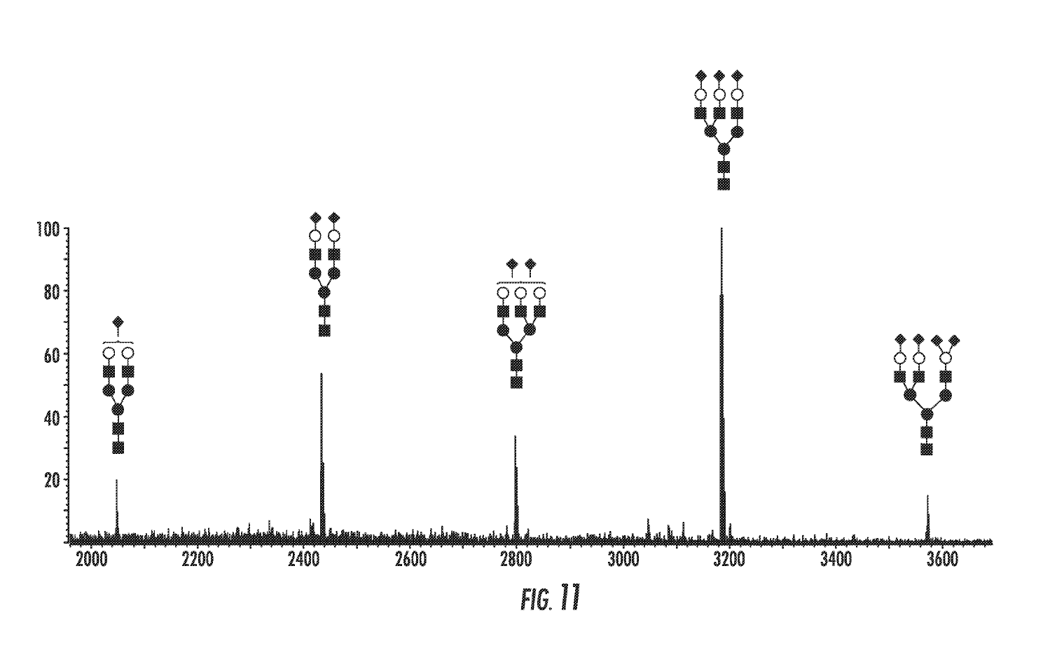

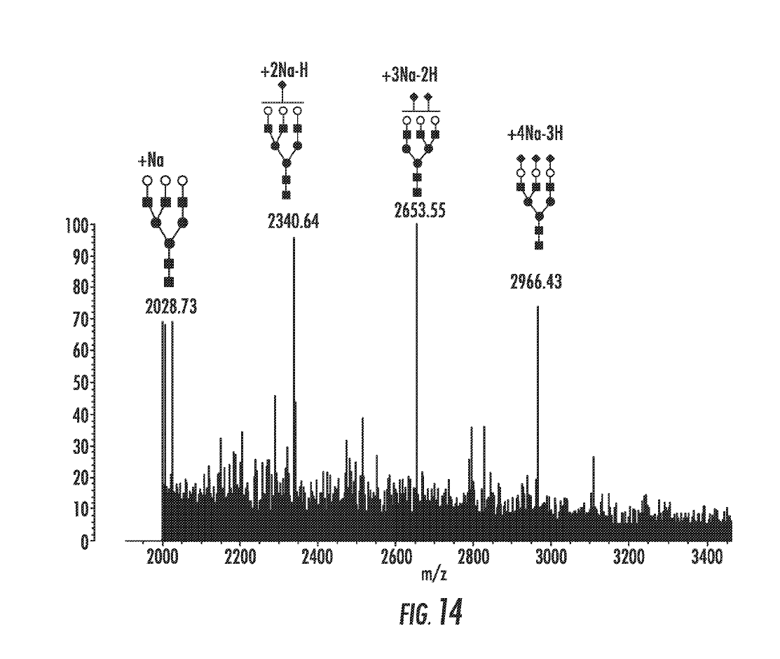

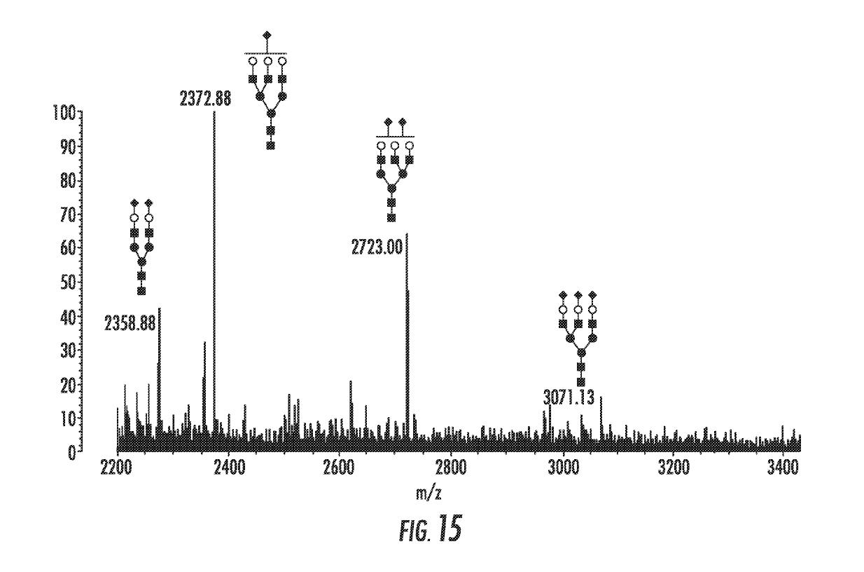

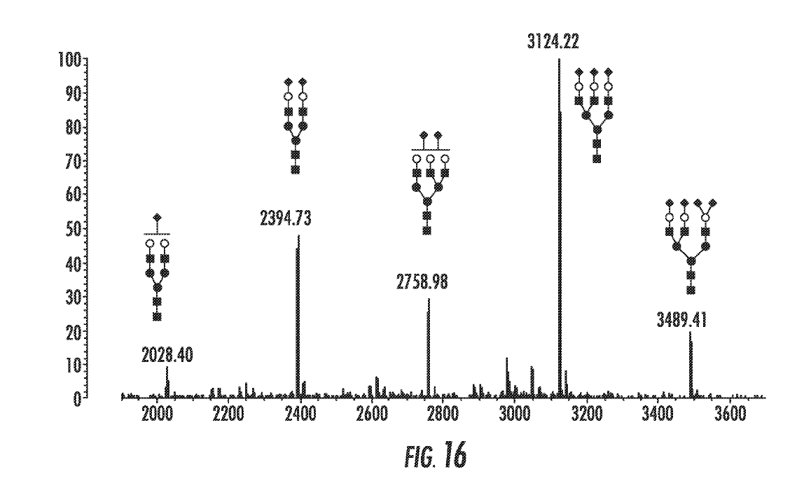

Fetuin was used as a model glycoprotein to develop method for analysis of the sialylated glycans. Fetuin consists of following previously reported sialylated glycans GlcNAc4Man3Gal2NeuNAc1, GlcNAc4Man3 Gal2NeuNAc2, GlcNAc5Man3Gal3NeuNAc2, GlcNAc5Man3Gal3NeuNAc3, and GlcNAc5Man3Gal3NeuNAc4. Sialic acid loss was observed when 10 μl of bovine unmodified fetuin N-glycans were analyzed using mass spectrometry with DHB/Dimethylaniline matrix. To stabilize and prevent sialic acid loss, it was necessary to modify sialic acids. Thus, amidation was used to modify the sialic acid in presence of EDC. To prevent protein loss, in the methods of the present invention, proteins were linked to solid support prior to the amidation reaction, which also aids in removal of impurities and changed reaction conditions with minimal sample loss. Once 10 μg of fetuin was conjugated on beads, amidation was used to modify sialic acids followed by releasing of N-glycans using PNGase F. Finally, glycans were analyzed using mass spectrometry (FIGS. 10, 14). We compared the amidation of sialylated glycans with acetohydrazide (FIG. 15) and aniline (FIG. 16). It was observed that the signal intensity of four fetuin sialylated glycans modified by acetohydrazide was not similar to previously published data with permethylated fetuin. However, sialylated glycans modified by aniline provided satisfactory results with similar sialylated glycan patterns as previously reported. Aniline makes the glycan more hydrophobic than acetohydrazide and hence aids the ionization of sialylated glycans enhancing their detection in MALDI. However, the mass of modified sialic acid with aniline is only 1.0744 Da apart from mass of Hexose+HexNAc, resulting in the overlap of peaks. This overlap was difficult to resolve by mass spectrometry for quantitative analysis. Hence, p-toluidine was used to prevent overlap between different glycans. p-Toluidine modification produced similar results as the aniline modification and all the previously reported fetuin sialylated glycans were observed (FIG. 11). Relative intensities of all the sialylated glycans were similar to previously observed by esterified or permethylated fetuin glycans with tri-antennary trisialylated glycan, which is the most abundant glycan (FIG. 11). Therefore, the loss of sialic acid was prevented with p-toluidine modification and detection of all the glycans was possible.

### Example 8

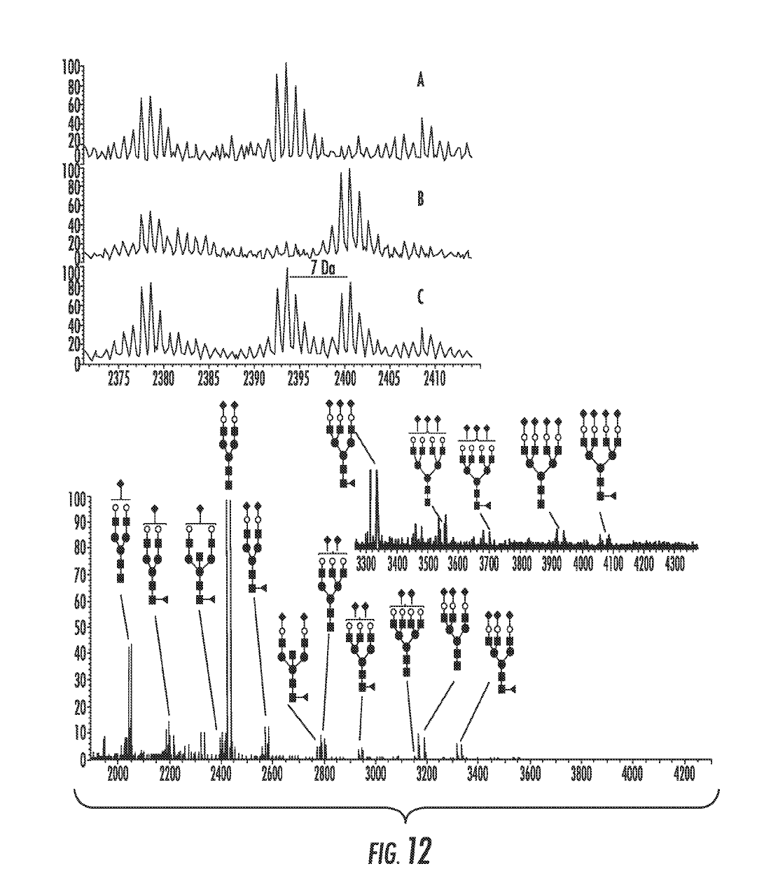

Detection of sialylated glycans from serum and determination of number of sialic acids in each glycan. We then applied the above method to analyze N-glycans from serum. In this case, we took two equal aliquots 1.6 μL of human serum and followed the protocol described above except the fact that one aliquot was labeled with heavy p-toluidine (D9) and other aliquot was labeled with light p-toludine. Glycans from each aliquot were analyzed with MS individually. If the m/z peak was observed in light labeled spectrum and absent in heavy labeled spectrum, then that m/z value was considered a sialylated glycan peak (FIG. 12A). If a peak observed in heavy labeled spectrum was absent in light labeled spectrum, and then such a peak was confirmed as signal from the same sialylated glycan (FIG. 12B). Serum glycans were also analyzed from each aliquot when mixed in 1:1 ratio (FIG. 12C). The differences between the peaks were used to determine the number of sialic acid present in the specific glycan (FIG. 12C). A difference of 7.06 Da between a light and heavy sialylated peak meant that one sialic acid had been modified by light and heavy p-toluidine thereby indicating the presence of one sialic acid on the glycan. A difference of 14.121 Da indicated that the glycan contained two modified sialic acids. Difference of a peak pair of differentially labeled peak pairs was searched for m/z values of multiple of 7 light-heavy ion pairs (FIG. 12D). A total of 45 N-glycans were identified from serum proteins and 21 sialylated N-glycan structures were successfully identified (Table 4). The sialylated glycan structures observed in our analysis were similar to results previously reported in serum. The 1:1 ratio observed in FIG. 12D also indicated the method could be used for quantitative analysis sialylated glycans.

TABLE 4
N-glycan compositions identified from serum. Two equal aliquots
of serum proteins were bound to beads and one aliquot was
labeled with light p-toluidine, second aliquot was labeled
with heavy p-toluidine. PNGase F released glycans were mixed
and analyzed using MALDI. The number of sialic acids present
on the glycans was identified by calculating the difference
between light and heavy labeled serum samples.

| | Fu- | | Hex- | Sialic | Theoretical | Observed |
|---|---|---|---|---|---|---|
| Core | cose | HexNAc | ose | Acid | Mass | Mass |
| Core + Na | 0 | 1 | 0 | 0 | 1136.423 | 1136.18 |
| Core + Na | 1 | 0 | 1 | 0 | 1241.438 | 1242.20 |
| Core + Na | 0 | 0 | 2 | 0 | 1257.433 | 1257.46 |
| Core + Na | 1 | 1 | 0 | 0 | 1282.481 | 1280.44 |
| Core + Na | 0 | 1 | 1 | 0 | 1298.475 | 1298.40 |
| Core + Na | 0 | 2 | 0 | 0 | 1339.518 | 1339.36 |
| Core + Na | 0 | 0 | 3 | 0 | 1419.486 | 1419.20 |
| Core + Na | 1 | 1 | 1 | 0 | 1444.533 | 1442.17 |
| Core + Na | 0 | 1 | 2 | 0 | 1460.528 | 1457.19 |
| Core + Na | 1 | 2 | 0 | 0 | 1485.576 | 1485.12 |
| Core + Na | 0 | 2 | 1 | 0 | 1501.571 | 1501.09 |
| Core + Na | 0 | 3 | 0 | 0 | 1542.613 | 1541.79 |
| Core + Na | 1 | 0 | 3 | 0 | 1565.544 | 1561.91 |
| Core + Na | 0 | 0 | 4 | 0 | 1581.538 | 1580.89 |
| Core + Na | 1 | 2 | 1 | 0 | 1647.629 | 1646.85 |
| Core + Na | 0 | 2 | 2 | 0 | 1663.624 | 1662.80 |
| Core + Na | 0 | 1 | 1 | 1 | 1678.741 | 1677.81 |
| Core + Na | 1 | 3 | 0 | 0 | 1688.671 | 1688.78 |
| Core + Na | 0 | 0 | 5 | 0 | 1743.591 | 1742.65 |
| Core + Na | 1 | 2 | 2 | 0 | 1809.582 | 1808.55 |
| Core + Na | 1 | 1 | 1 | 1 | 1824.799 | 1822.34 |
| Core + Na | 1 | 3 | 1 | 0 | 1850.724 | 1850.32 |
| Core + Na | 0 | 2 | 1 | 1 | 1881.836 | 1881.42 |
| Core + Na | 0 | 0 | 6 | 0 | 1905.644 | 1904.35 |
| Core + Na | 1 | 0 | 3 | 1 | 1945.809 | 1942.84 |
| Core + Na | 1 | 3 | 2 | 0 | 2012.777 | 2011.17 |
| Core + Na | 1 | 2 | 1 | 1 | 2027.894 | 2026.16 |
| Core + Na | 0 | 2 | 2 | 1 | 2043.889 | 2043.12 |
| Core + Na | 1 | 2 | 2 | 1 | 2189.947 | 2189.38 |
| Core + Na | 1 | 4 | 2 | 0 | 2215.872 | 2215.74 |
| Core + Na | 0 | 3 | 2 | 1 | 2246.85 | 2247.12 |
| Core + Na | 0 | 7 | 0 | 0 | 2354.995 | 2354.49 |
| Core + Na | 1 | 3 | 2 | 1 | 2393.042 | 2393.41 |
| Core + Na | 0 | 2 | 2 | 2 | 2424.154 | 2424.43 |
| Core + Na | 1 | 2 | 2 | 2 | 2570.212 | 2570.26 |
| Core + Na | 1 | 3 | 2 | 2 | 2773.308 | 2773.26 |
| Core + Na | 0 | 3 | 3 | 2 | 2789.303 | 2788.29 |
| Core + Na | 1 | 3 | 3 | 2 | 2935.361 | 2935.23 |
| Core + Na | 0 | 4 | 4 | 2 | 3154.451 | 3154.69 |
| Core + Na | 0 | 3 | 3 | 3 | 3169.568 | 3169.30 |
| Core + Na | 1 | 3 | 3 | 3 | 3315.626 | 3315.28 |
| Core + Na | 0 | 4 | 4 | 3 | 3534.716 | 3534.28 |
| Core + Na | 1 | 4 | 4 | 3 | 3680.774 | 3680.18 |
| Core + Na | 0 | 4 | 4 | 4 | 3914.982 | 3914.23 |
| Core + Na | 1 | 4 | 4 | 4 | 4061.040 | 4061.23 |

Observed mass is the mass of glycan labeled with light p-toluidine, core represents the core structure of the N-glycan, which is 2HexNAc and 3 Hexose.

### Example 9

### Quantitative Analysis of Sialylated N-Glycans from Cells Treated with ManNAc

### Changes of N-Glycan Sialylation Driven by Metabolic Flux

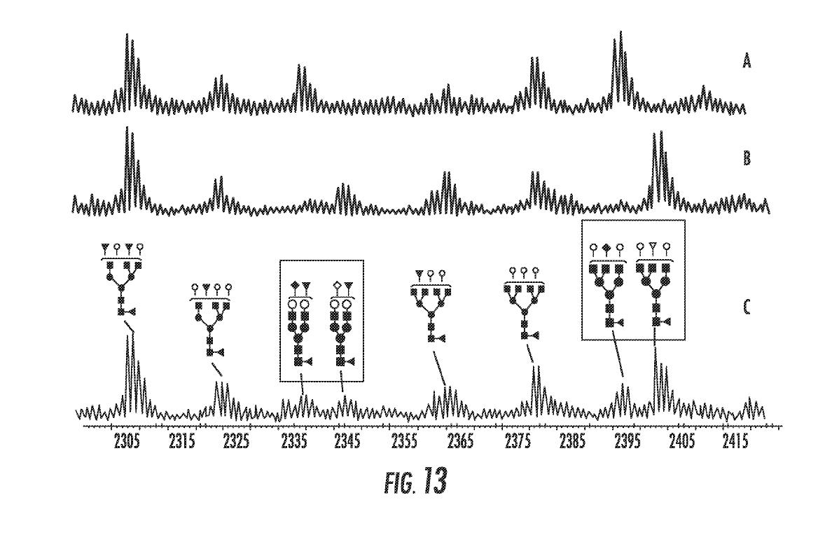

It has been well established that the extent of glycan sialylation depends on sialyltransferases, the biosynthetic enzymes responsible for adding a sialic acid to an underlying galactose or GalNAc residue. However, we recently showed that N-glycan sialylation also depends on metabolic flux through the sialic acid biosynthetic pathway. In a previous study we identified glycopeptides that were affected by flux changes in flux but did not analyze the structures of the glycans themselves. To fill this void, in the present invention we profiled N-glycans in SW1990 treated with 1,3,4-O-Bu3ManNAc as previously described. Protein extract from 1,3,4-O-Bu3ManNAc treated SW1990 cells and untreated control cells were conjugated to solid support and labeled with isotopic p-toluidine as described above. The mass spectrometric analysis of labeled glycans identified 87 N-glycans based on accurate mass (data not shown). Sialic acid compositional assignment was determined based on the differences of heavy and light labeled glycans. Twenty one heavy and light glycan peaks were identified with a mass shift of 7 or a multiple of 7, confirming that the presence of 21 sialylated glycans (FIGS. 13A and 13B). Qualitative analysis of N-glycans from cells with and without ManNAc treatment using isotopic labeling of sialylated N-glycans with 1:1 combined samples identified and quantified 14 sialylated glycans (Table 5). Seven other sialylated Nglycans could not be quantified due to low signal to noise ratio when the two samples were combined for MS analysis. The quantitative results showed that while the most Nglycans showed minor or no obvious differences between ManNAc treated and untreated cells (FIG. 13C), six sialylated N-glycan compositions exhibited a significant increase in abundance, specifically the mono-sialylated tertra-antennary N-glycan (Table 5). There was no significant decrease in sialylated N-glycans when cells were treated with 1,3,4-O-Bu3ManNAc, indicating metabolic flux contributed to the overall extent of sialylation on the proteins.

TABLE 5
Relative Quantitation of Sialylated N-glycans Identified from
SW1990 Cells with and without 1,3,4-O—Bu3ManNAc
Treatment Glycans from cells without treatment were labeled with light
p-toluidine, glycans from cells with treatment were labeled with heavy
p-toluidine. PNGase F released glycans were analyzed using MALDI.
The number of sialic acids present on the glycans was identified by
calculating the difference between light and heavy labeled serum
samples and quantified based on their intensities.

| | Fu- | | Hex- | Sialic | | | |
|---|---|---|---|---|---|---|---|
| Core + Na | cose | HexNAc | ose | Acid | [M + NA]* | H/L | Stdev |
| Core + Na | 0 | 2 | 2 | 1 | 2043.89 | 1.29 | 0.44 |
| Core + Na | 1 | 2 | 2 | 1 | 2189.95 | 1.09 | 0.17 |
| Core + Na | 2 | 2 | 2 | 1 | 2336.01 | 1.10 | 0.13 |
| Core + Na | 1 | 3 | 2 | 1 | 2393.04 | 1.95 | 0.11 |
| Core + Na | 2 | 3 | 2 | 1 | 2539.10 | 1.31 | 0.10 |
| Core + Na | 1 | 3 | 3 | 1 | 2555.10 | 1.31 | 0.45 |
| Core + Na | 1 | 2 | 2 | 2 | 2570.21 | 1.02 | 0.13 |
| Core + Na | 3 | 3 | 2 | 1 | 2585.15 | 1.00 | 0.17 |
| Core + Na | 2 | 3 | 3 | 1 | 2701.15 | 1.03 | 0.23 |
| Core + Na | 1 | 4 | 3 | 1 | 2758.19 | 1.66 | 0.31 |
| Core + Na | 1 | 3 | 2 | 2 | 2773.31 | 1.70 | 0.18 |
| Core + Na | 2 | 4 | 3 | 1 | 2904.25 | 2.04 | 0.50 |
| Core + Na | 1 | 3 | 3 | 2 | 2935.36 | 0.98 | 0.21 |
| Core + Na | 3 | 4 | 3 | 1 | 3050.31 | 1.39 | 0.30 |

H: Heavy p-toluidine labeled sialylated N-glycans from 1,3,4-O—Bu3ManNAc treated cells.
L: Light p-toluidine labeled sialylated N-glycans from untreated (control) cells.
Core represents core structure of N-glycan which is 2 HexNAc and 3 Hexose.

The methods of the present invention provide novel chemical derivatization strategies that stabilize the sialylated glycans and prevents loss of sialic acid. Specifically, the labeling of sialic acids with p-toluidine makes glycans hydrophobic and allows retention on C18 columns and improves ionization of glycans. The isotopic labeling with the 7 Da difference per sialic acid in the same sialylated glycan structure from two different samples is substantial enough to provide non-overlapping pairs compared to other low mass shift techniques and hence improves quantitation efficiency. The difference between the pair also helps definitively determine the number of sialic acids present in the glycan. Using solid-phase sialic acid labeling, the sample is first bound to the beads, and all the subsequent steps result in minimum sample loss. Sialic acid labeling is performed at protein level without processing and removal of proteins. The labeled samples are combined and processed through the rest of sample preparation steps, which reduces the errors due to sample handling in all the subsequent steps such as PNGase F digestion and sample clean up. Along with sialic acids on glycans, aspartic acids and glutamic acids on proteins are also modified and can be used for peptide/protein quantitation from the same specimens upon releasing peptides from solid support using proteolysis.

### Example 10

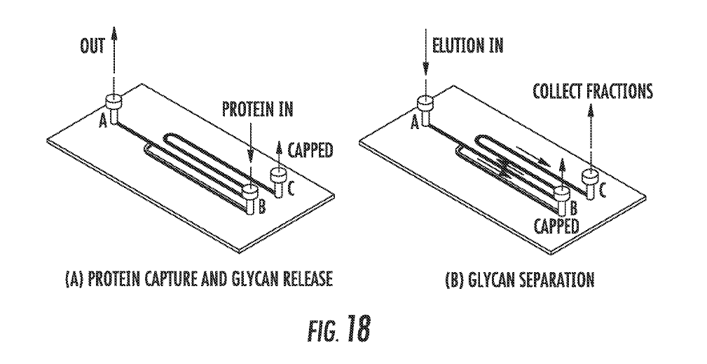

Solid-Phase Glycan Extraction on chip-LC apparatus. Briefly, in an embodiment, proteins (20 μL RNase B at 10 μg/μL; 20 μL human serum; or 400 μg mouse serum or tissue proteins) were denatured in a total of 200 μL of binding buffer (40 mM sodium citrate and 20 mM sodium carbonate, pH 10) at 100° C. for 10 minutes AminoLink beads packed in the microchip of the present invention were washed with 400 μL binding buffer (pH 10) at a flow rate of 20 μL/min (note: the flow rate is 20 μL/min unless specifically mentioned) using syringe pump (Pump 11 Elite Infusion/Withdrawal Programmable Dual Syringe; Harvard Apparatus; Holliston, Mass.). Port C is then capped and with port A and B open, and a protein solution was then infused into the second channel in the direction of port B to A and incubated for 2 hours to conjugate proteins to AminoLink beads (FIG. 18A). The conjugated proteins were then reduced by NaCNBH₃ for 2 hours by infusing 50 mM NaCNBH₃ in 1×PBS in the second channel from port B to A. The free aldehyde groups were further blocked by infusion of 1 M Tris-HCl in the presence of 50 mM NaCNBH₃ in the second channel from port B to A. To prepare solution for sialic acid modification, p-toluidine (47 mg) was dissolved in 367 μL DI and 33 μL HCl (36-38%). Then EDC (40 μL) and HCl (25 μL) were added in 400 μL p-toluidine solution, whose final pH was adjusted to 4-6 by EDC (increase pH) or HCl (decrease pH). The p-toluidine solution (460 μL) was then infused into the second channel from port B to A and incubated for 3 hours. After washing the second channel from port B to A with water, PNGase F (4 μL) was injected into the second channel after mixing with 4 μL G7 buffer (New England Biolabs) in 32 μL DI water, and incubated for 2 hours at 37° C. All needles were then capped to avoid buffer evaporation during incubation.

### Example 11

Chip-LC using Reverse-Phase Porous Graphitized Carbon. To condition porous graphitized carbons (PGC) in the first channel, port B was capped while 200 μL of 80% acetonitrile (0.1% FA) was flushed through the first channel in the direction of port A to C. Then 400 μL of 0.1% FA was then injected through the first channel (FIG. 18B). This step was performed before proteins were immobilized to the beads described in Example 10. Following the glycan release from solid support in Example 10, washing solution (400 μL, 0.1% formic acid (FA)) was injected into port B while C was capped. The released glycans become enriched at the intersection between the first and second channel (5) for further separation. Port B was then capped while port A and C are opened for collecting elution fractions. The eluate consisted of 0.1% FA mobile phase in a variety of solutions (gradient) consisting of acetonitrile and HPLC grade water, starting from 0% acetonitrile up to 80%. The details of acetonitrile concentration used are given in the examples below.

### Example 12

MALDI-TOF MS Analysis. Glycan fractions eluted from microchip of the present invention at port C were analyzed by AXIMA Resonance mass spectrometer (Shimadzu; Columbia, Md.). The MALDI matrix was prepared by mixing 4 μL DMA in 200 μL DHB (100 μg/μL in 50% acetonitrile, 0.1 mM NaCl) to increase the ionization of glycans. This matrix can form uniform crystals and improve glycan signal by enhancement of laser power absorption and ionization efficiency. The laser power was set to 100 for 2 shots each in 100 locations per spot.

### Example 13

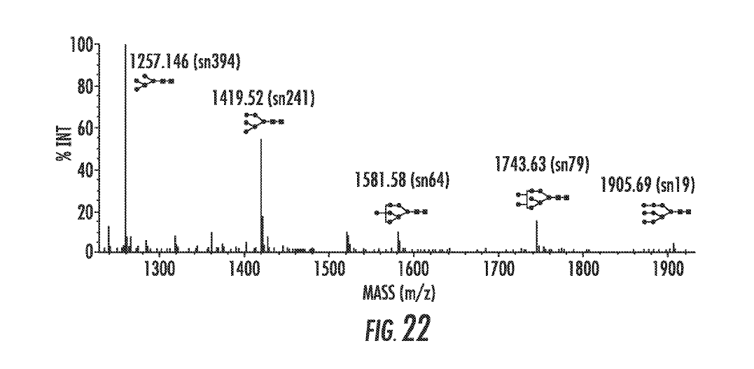

As a proof-of-concept, a standard glycoprotein, RNase B, was used for on-chip protein capture, glycan enzymatic release and purification using PGC before detection by MS. To demonstrate the performance of GIG on the microfluidic chip of the present invention, 100 μL of beads (AminoLink Resin) were packed in the second channel of the microchip. RNase B (200 μg) was conjugated to AminoLink beads and released N-glycans were purified and eluted from PGC in the first channel using 40 μL 80% acetonitrile, 0.1% FA. All five N-glycans extracted from RNase B were observed (FIG. 22). Five N-glycans, Man5 to Man9, were observed according to their accurate masses. Consistent with a previous report, Man5 was the most dominant oligomannose from RNase B. These results indicate that solid-phase protein capture and glycan release and purification on the chip of the present invention is feasible for N-glycan analysis.

After glycans were captured and released using the GIG portion of the chip, the chip-LC portion, having the first channel packed with PGC, should further improve the performance of glycan analysis by glycan purification and LC separation. We thus applied the GIG-chipLC apparatus of the present invention to extract N-glycans from complex samples, mouse blood serum (MBS) and mouse heart tissue (MT). To stabilize sialic acids and improve their hydrophobicity for MS detection, p-toluidine was used to modify sialylated glycans on solid-phase before glycan release as described herein.

### Example 14

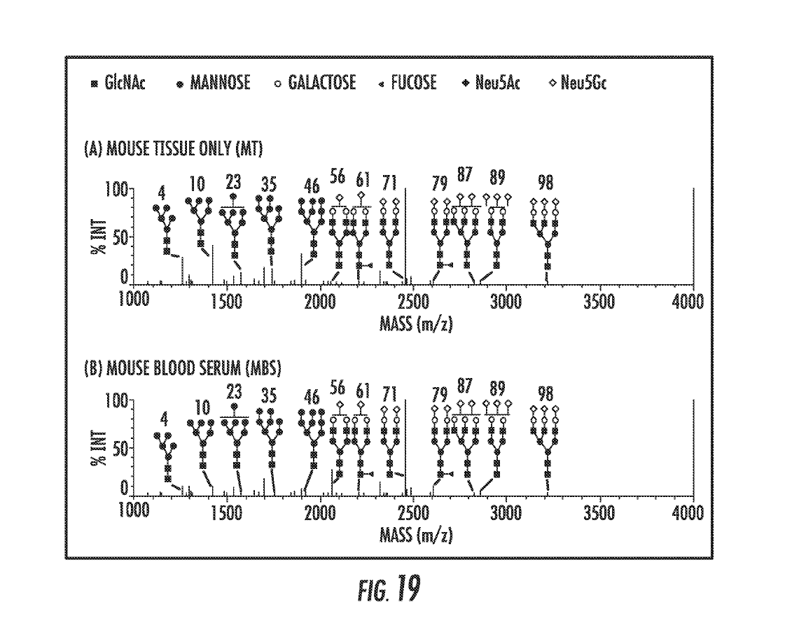

MBS and MT were first sonicated for 30 seconds in RIPA buffer at an interval of 30 seconds on ice for 3 minutes. The loading for each sample was 400 μg and sialylated glycans were modified before release of N-glycans. Glycans were analyzed by MS without, and with chip-LC separation. When the released glycans were analyzed without chip-LC separation, the PGC component was used as glycan purification cartridge and the glycans were eluted from the column with 80% acetonitrile in one fraction as described above for RNase B analysis. Based on the MS analysis of the released glycans from mouse serum and heart tissue, several interesting results were observed. First, oligomannoses and complex N-glycans were detected in both serum and heart tissue. Second, tissue contained higher level of oligomannoses than those from mouse blood serum (FIG. 19 (A) vs. (B)). Third, the level of sialylated glycans was higher in mouse blood serum compared to those from heart tissue. Some of those abundant sialylated glycans have been previously reported in mouse serum, e.g., bi- or tri-antennary Nen5Gc glycans. Overall, without LC separation, glycans were detected in both MT and MBS. However, tissue- or serum-specific glycans, likely presented in low abundance not detected in these spectra, might be detectable when the glycans were separated prior to MS analysis.

### Example 15

To determine whether chip-LC separation allowed detection of specific glycans from heart tissue or blood serum, the extracted N-glycans from MBS and MT described above were separated by use of the chip-LC portion of the apparatus using stepwise acetonitrile elution gradient (1% step from 0-80%) with 40 μL/fraction (flow rate: 10 μL/min) Each fraction was analyzed using MS. We observed 65 distinct N-glycan masses commonly detected in both MBS and MT, including most fucosylated and sialylated glycans. The complete glycan list is given in Table 6. By use of the GIG-chip-LC apparatus of the present invention, we detected 96 N-glycan masses in MBS and 72 N-glycan masses in MT. Seven N-glycan masses were only detected in MT, while 31 N-glycan masses were only detected from MBS. These results show that the novel microfluidic device with integrated GIG and chip-LC facilitates the identification of tissue-specific glycans that might be relevant to the physiological or pathological status of the tissue.

TABLE 6
N-glycan masses and proposed glycan composition detected in mouse blood serum
and heart tissue (note: composition for N-glycan core is not included in the
table). Glycans were detected by MALDI-MS using Shimadzu AXIMA Resonance.

| Glycan | Fucose | HexNAc | Hexose | NeuAc | NeuGc | Xylose | Sulphate | MS (Da) | MBS | MT |
|---|---|---|---|---|---|---|---|---|---|---|
| 1 | 0 | 0 | 0 | 0 | 0 | 0 | 0 | 933.1 | + | + |
| 2 | 1 | 0 | 0 | 0 | 0 | 0 | 0 | 1079.1 | + | + |
| 3 | 0 | 0 | 1 | 0 | 0 | 0 | 0 | 1095.1 | + | + |
| 4 | 0 | 1 | 0 | 0 | 0 | 0 | 0 | 1136.2 | + | + |
| 5 | 0 | 0 | 2 | 0 | 0 | 0 | 0 | 1257.1 | + | + |
| 6 | 1 | 1 | 0 | 0 | 0 | 0 | 0 | 1282.2 | + | + |
| 7 | 0 | 1 | 1 | 0 | 0 | 0 | 0 | 1298.2 | + | + |
| 8 | 0 | 2 | 0 | 0 | 0 | 0 | 0 | 1339.2 | + | + |
| 9 | 1 | 0 | 2 | 0 | 0 | 0 | 0 | 1403.1 | + | + |
| 10 | 0 | 0 | 3 | 0 | 0 | 0 | 0 | 1419.2 | + | + |
| 11 | 1 | 1 | 0 | 0 | 0 | 0 | 1 | 1442.2 | + | + |
| 12 | 1 | 1 | 1 | 0 | 0 | 0 | 0 | 1444.2 | + | + |
| 13 | 0 | 1 | 2 | 0 | 0 | 0 | 0 | 1460.3 | + | + |
| 14 | 1 | 2 | 0 | 0 | 0 | 0 | 0 | 1485.2 | + | + |
| 15 | 0 | 2 | 1 | 0 | 0 | 0 | 0 | 1501.2 | + | + |
| 16 | 2 | 1 | 0 | 0 | 0 | 0 | 1 | 1508.1 | + | |
| 17 | 0 | 1 | 0 | 1 | 0 | 0 | 0 | 1516.1 | + | |
| 18 | 0 | 0 | 2 | 0 | 0 | 2 | 0 | 1522.1 | | + |
| 19 | 0 | 1 | 0 | 0 | 1 | 0 | 0 | 1532.1 | + | + |
| 20 | 0 | 3 | 0 | 0 | 0 | 0 | 0 | 1542.1 | + | + |
| 21 | 0 | 1 | 1 | 0 | 0 | 2 | 0 | 1562.2 | + | + |
| 22 | 3 | 1 | 0 | 0 | 0 | 0 | 0 | 1574.2 | + | |
| 23 | 0 | 0 | 4 | 0 | 0 | 0 | 0 | 1581.2 | + | + |
| 24 | 1 | 1 | 2 | 0 | 0 | 0 | 0 | 1606.3 | + | + |
| 25 | 1 | 1 | 2 | 0 | 0 | 0 | 0 | 1622.2 | + | + |
| 26 | 1 | 2 | 1 | 0 | 0 | 0 | 0 | 1647.2 | + | + |
| 27 | 0 | 2 | 2 | 0 | 0 | 0 | 0 | 1663.2 | + | + |
| 28 | 0 | 1 | 1 | 1 | 0 | 0 | 0 | 1678.0 | + | |
| 29 | 0 | 0 | 3 | 0 | 0 | 2 | 0 | 1684.1 | + | + |
| 30 | 1 | 3 | 0 | 0 | 0 | 0 | 0 | 1688.2 | + | + |
| 31 | 0 | 1 | 1 | 0 | 1 | 0 | 0 | 1694.2 | + | + |
| 32 | 0 | 3 | 1 | 0 | 0 | 0 | 0 | 1704.3 | | |
| 33 | 0 | 1 | 2 | 0 | 0 | 2 | 0 | 1725.2 | | |
| 34 | 3 | 1 | 1 | 0 | 0 | 0 | 0 | 1737.2 | + | |
| 35 | 0 | 0 | 5 | 0 | 0 | 0 | 0 | 1743.3 | + | + |
| 36 | 3 | 1 | 0 | 0 | 0 | 0 | 0 | 1766.1 | | + |
| 37 | 0 | 1 | 2 | 0 | 0 | 2 | 1 | 1804.1 | | + |
| 38 | 1 | 2 | 1 | 0 | 0 | 0 | 0 | 1809.2 | + | + |
| 39 | 1 | 1 | 1 | 1 | 0 | 0 | 0 | 1823.0 | + | + |
| 40 | 0 | 2 | 3 | 0 | 0 | 0 | 0 | 1825.1 | + | + |
| 41 | 1 | 1 | 1 | 0 | 1 | 0 | 0 | 1840.4 | + | + |
| | 0 | 1 | 2 | 1 | 0 | 0 | 0 | | | |
| 42 | 1 | 3 | 1 | 0 | 0 | 0 | 0 | 1850.3 | | + |
| 43 | 0 | 1 | 2 | 0 | 1 | 0 | 0 | 1856.3 | + | + |
| 44 | 0 | 0 | 1 | 0 | 2 | 0 | 0 | 1887.1 | | + |
| | 0 | 3 | 0 | 0 | 0 | 2 | 1 | | | |
| 45 | 0 | 2 | 1 | 0 | 2 | 0 | 0 | 1898.2 | + | |
| 46 | 0 | 0 | 6 | 0 | 0 | 0 | 0 | 1905.2 | + | + |
| 47 | 1 | 2 | 3 | 0 | 0 | 0 | 0 | 1971.1 | + | + |
| 48 | 2 | 1 | 1 | 0 | 1 | 0 | 0 | 1987.3 | | |
| | 0 | 2 | 4 | 0 | 0 | 0 | 0 | | | |
| 49 | 2 | 3 | 0 | 0 | 0 | 0 | 2 | 1994.1 | + | + |
| 50 | 1 | 1 | 2 | 0 | 1 | 0 | 0 | 2002.2 | + | + |
| 51 | 1 | 3 | 2 | 0 | 0 | 0 | 0 | 2012.2 | + | + |
| 52 | 0 | 1 | 3 | 0 | 1 | 0 | 0 | 2018.3 | + | + |
| 53 | 0 | 3 | 3 | 0 | 0 | 0 | 0 | 2028.3 | + | |
| 54 | 1 | 1 | 0 | 2 | 0 | 0 | 0 | 2041.3 | + | + |
| 55 | 1 | 2 | 1 | 0 | 1 | 0 | 0 | 2043.2 | + | + |
| | 0 | 2 | 2 | 1 | 0 | 0 | 0 | | | |
| 56 | 0 | 2 | 2 | 0 | 1 | 0 | 0 | 2059.3 | + | + |
| 57 | 0 | 0 | 7 | 0 | 0 | 0 | 0 | 2067.1 | + | + |
| 58 | 1 | 2 | 4 | 0 | 0 | 0 | 0 | 2133.0 | + | + |
| 59 | 1 | 3 | 3 | 0 | 0 | 0 | 0 | 2174.3 | | |
| 60 | 1 | 2 | 2 | 1 | 0 | 0 | 0 | 2189.3 | + | + |
| | 2 | 2 | 1 | 0 | 1 | 0 | 0 | | | |
| 61 | 1 | 2 | 2 | 0 | 1 | 0 | 0 | 2205.3 | + | + |
| | 0 | 2 | 3 | 1 | 0 | 0 | 0 | | | |
| 62 | 0 | 2 | 3 | 1 | 0 | 0 | 0 | 2222.1 | + | |
| 63 | 0 | 3 | 2 | 1 | 0 | 0 | 0 | 2247.1 | + | |
| | 1 | 3 | 1 | 0 | 1 | 0 | 0 | | | |
| 64 | 0 | 3 | 2 | 0 | 1 | 0 | 0 | 2263.0 | + | |
| 65 | 2 | 1 | 3 | 1 | 0 | 0 | 0 | 2294.2 | + | |
| | 3 | 1 | 2 | 0 | 1 | 0 | 0 | | | |
| 66 | 1 | 2 | 3 | 1 | 0 | 0 | 0 | 2351.1 | + | + |
| | 2 | 2 | 2 | 0 | 1 | 0 | 0 | | | |
| 67 | 0 | 7 | 0 | 0 | 0 | 0 | 0 | 2354.3 | + | + |
| 68 | 0 | 2 | 4 | 1 | 0 | 0 | 0 | 2367.1 | + | + |
| | 1 | 2 | 3 | 0 | 1 | 0 | 0 | | | |
| 69 | 0 | 2 | 2 | 2 | 0 | 0 | 0 | 2424.3 | + | + |
| | 0 | 3 | 3 | 0 | 1 | 0 | 0 | | | |
| 70 | 0 | 2 | 2 | 1 | 1 | 0 | 0 | 2440.0 | + | + |
| 71 | 0 | 2 | 2 | 0 | 2 | 0 | 0 | 2456.2 | + | + |
| 72 | 0 | 4 | 3 | 0 | 0 | 0 | 3 | 2471.3 | + | + |
| 73 | 3 | 2 | 2 | 1 | 0 | 0 | 0 | 2483.3 | + | + |
| 74 | 3 | 2 | 2 | 0 | 1 | 0 | 0 | 2498.0 | + | + |
| 75 | 1 | 3 | 3 | 1 | 0 | 0 | 0 | 2556.0 | | + |
| | 2 | 3 | 2 | 0 | 1 | 0 | 0 | | | |
| 76 | 1 | 2 | 2 | 2 | 0 | 0 | 0 | 2569.0 | + | + |
| 77 | 1 | 2 | 2 | 1 | 1 | 0 | 0 | 2585.0 | + | + |
| 78 | 3 | 6 | 0 | 0 | 0 | 0 | 0 | 2591.0 | + | |
| 79 | 1 | 2 | 2 | 0 | 2 | 0 | 0 | 2602.2 | + | + |
| 80 | 0 | 4 | 3 | 1 | 0 | 0 | 0 | 2611.2 | + | |
| 81 | 0 | 3 | 2 | 0 | 2 | 0 | 0 | 2660.0 | + | + |
| 82 | 3 | 5 | 2 | 0 | 0 | 0 | 0 | 2712.1 | + | |
| 83 | 2 | 3 | 3 | 0 | 1 | 0 | 0 | 2718.0 | + | |
| 84 | 4 | 6 | 0 | 0 | 0 | 0 | 0 | 2738.0 | + | |
| 85 | 0 | 6 | 4 | 0 | 0 | 0 | 0 | 2801.9 | + | |
| 86 | 0 | 3 | 3 | 1 | 1 | 0 | 0 | 2806.9 | + | |
| 87 | 0 | 3 | 3 | 0 | 2 | 0 | 0 | 2820.4 | + | + |
| 88 | 3 | 4 | 4 | 0 | 0 | 0 | 0 | 2833.9 | + | |
| 89 | 0 | 2 | 2 | 0 | 3 | 0 | 0 | 2851.4 | + | + |
| 90 | 3 | 2 | 2 | 1 | 1 | 0 | 0 | 2878.0 | + | + |
| 91 | 1 | 3 | 3 | 1 | 1 | 0 | 0 | 2951.4 | + | |
| | 2 | 3 | 2 | 0 | 2 | 0 | 0 | | | |
| 92 | 3 | 7 | 1 | 0 | 0 | 0 | 0 | 2955.9 | + | |
| 93 | 1 | 3 | 3 | 0 | 2 | 0 | 0 | 2967.3 | + | |
| 94 | 1 | 2 | 2 | 0 | 3 | 0 | 0 | 2997.4 | + | + |
| 95 | 3 | 6 | 3 | 0 | 0 | 0 | 0 | 3076.8 | + | |
| 96 | 3 | 7 | 2 | 0 | 0 | 0 | 0 | 3117.0 | + | |
| 97 | 3 | 5 | 5 | 0 | 0 | 0 | 0 | 3198.9 | + | |
| 98 | 0 | 3 | 3 | 0 | 3 | 0 | 0 | 3216.4 | + | + |
| 99 | 1 | 3 | 3 | 3 | 0 | 0 | 0 | 3314.3 | + | |
| 100 | 1 | 3 | 3 | 2 | 1 | 0 | 0 | 3331.3 | | + |
| | 1 | 4 | 4 | 0 | 2 | 0 | 0 | | | |
| 101 | 1 | 8 | 4 | 0 | 0 | 0 | 0 | 3352.9 | + | |
| 102 | 1 | 3 | 3 | 0 | 3 | 0 | 0 | 3362.4 | + | + |
| 103 | 0 | 4 | 4 | 0 | 3 | 0 | 0 | 3582.8 | + | |
| 104 | 0 | 3 | 3 | 0 | 4 | 0 | 0 | 3613.4 | + | + |
| 105 | 1 | 3 | 3 | 0 | 4 | 0 | 0 | 3759.2 | + | |
| 106 | 0 | 4 | 4 | 0 | 4 | 0 | 0 | 3978.8 | + | |
| 107 | 1 | 4 | 4 | 0 | 4 | 0 | 0 | 4124.7 | + | |

(+ present; − not present)

Several studies have focused on the identification of disease-associated glycan changes in blood serum due to its easy accessibility and noninvasive diagnosis, however, blood serum likely contains a large number of N-glycans, which are secreted from different organs. Alternatively, it might be more informative to study glycans from specific tissue of interest for organ-specific glycan changes. Therefore, specific glycan analysis of disease affected tissues should be an effective means for identification of abnormal glycans associated with a specific disease.

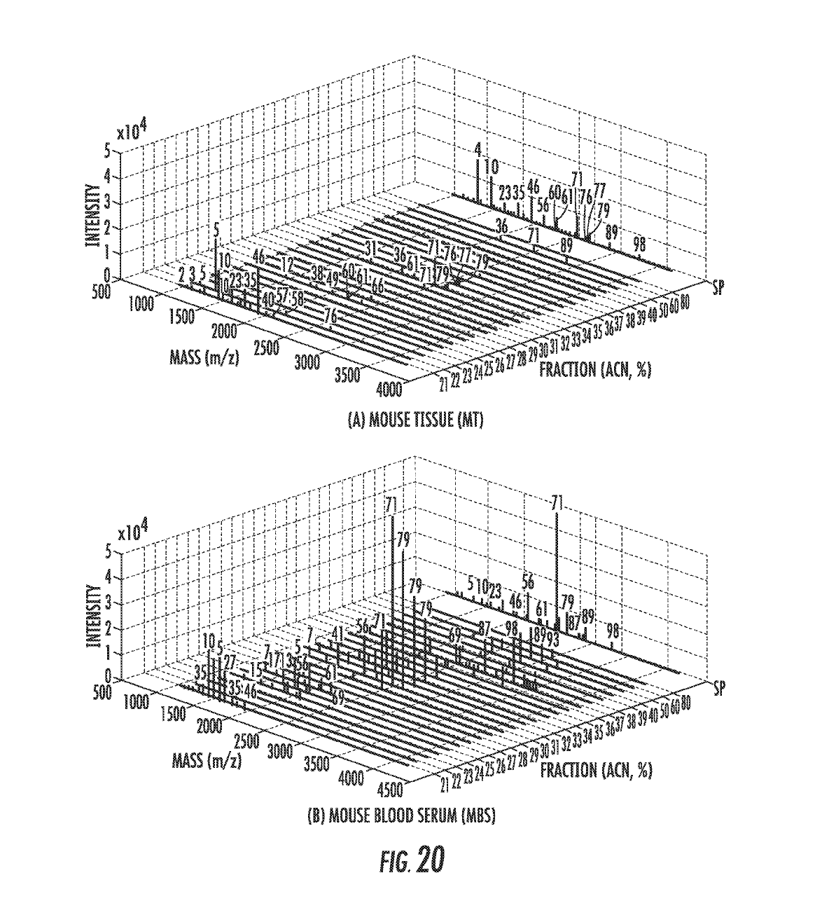

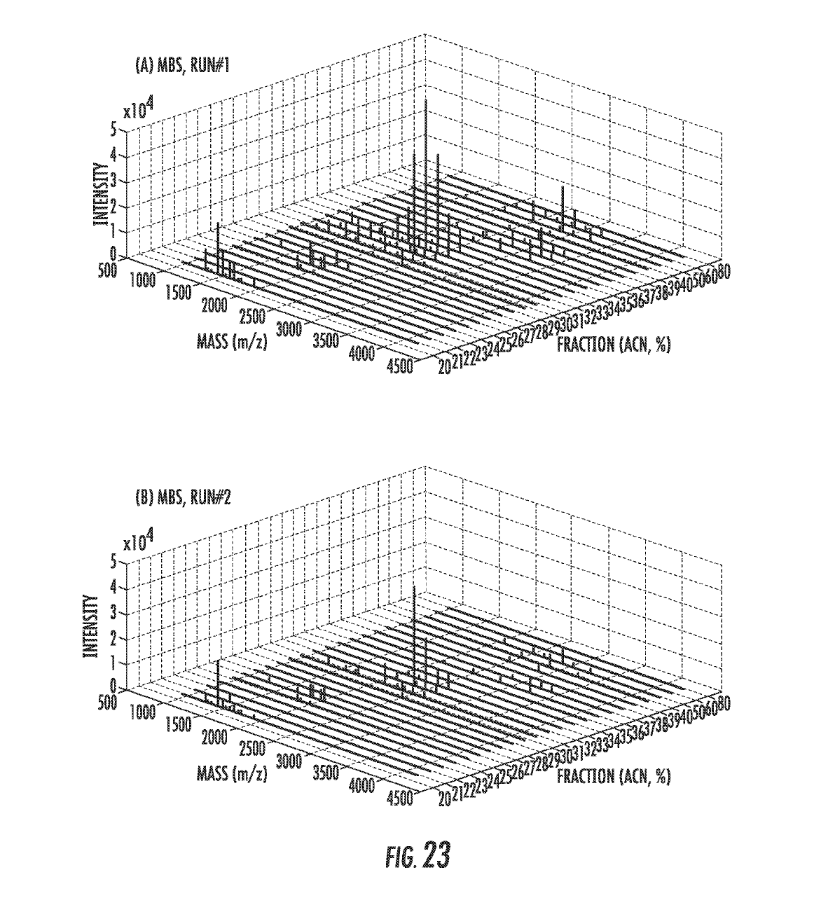

The N-glycans eluted in different percentage of acetonitrile by chip-LC fraction were compared with the N-glycans without chip-LC separation in a pseudo-3D plot (FIG. 20; peak number corresponding to the glycan listed in Table 6). The high-abundance N-glycans, which were detected by MALDI-MS without chip-LC fraction, were also prominent after chip-LC separation, including oligomannoses, bi- and tri-antennary sialic acids (FIG. 20). Oligomannoses were dominant N-glycans in mouse heart tissue (FIG. 20A); while the sialylated glycans were mostly abundant in mouse blood serum (FIG. 20B). Remarkably, chip-LC was highly reproducible on glycan fractionation (FIG. 23). For example, Man5-Man9 glycans with masses lower than 2000 Da were eluted in fraction of 21% acetonitrile across different sample processes, while biantennary sialylated glycan (71) was eluted in 30% acetonitrile fraction and triantennary sialylated glycan (98) was detected at 37% acetonitrile fraction.

The reproducible glycan elution in specific acetonitrile fraction from chip-LC also indicated that the GIG-chip-LC apparatus of the present invention may be able to separate glycan isomers with the same mass (FIG. 20). For example, oligomannoses (5, 10, 23, 46, 56), were co-eluted in 21% acetonitrile. However, we found that a high intensity peak (5) was also eluted in 22% of acetonitrile, as well as a small amount of peak 10 and 23 in the same fraction (22%). These results could not have resulted from the incomplete elution of Man5-Man7 glycans by 21% acetonitrile since no glycans were detected at the end of 40 μL/fraction elution (flow rate: 10 μL/min) Similarly, biantennary sialic acid (71) was observed from fraction 30% to 34% and 37%, but not between 34% and 36% (FIG. 20B). These results indicate that isomers of these glycans were probably eluted in different fractions. According to detailed study on oligomannose structures by tandem MS analysis, Man5, Man6, and Man7 have several isomers. For example, Man5 from RNase B has potentially four isomers, including one triantennary and three biantennary structures. It is possible that different isomers are eluted in 21% or 22% of acetonitrile. Similarly, Man6 isomers are eluted in 21% and 22% fractions. Additional studies would be needed to determine the identities of different oligomannose isomers after glycan permethylation, since PGC has good selective interactions with methyl substituted groups to further increase glycan retention. In addition, permethylated glycans could be used for detail structural analysis. Investigation on oligomannose isomers could be very useful to understanding the changes of oligomannoses in diseases such as the alterations of gp120 glycosylation in HIV.

### Example 16

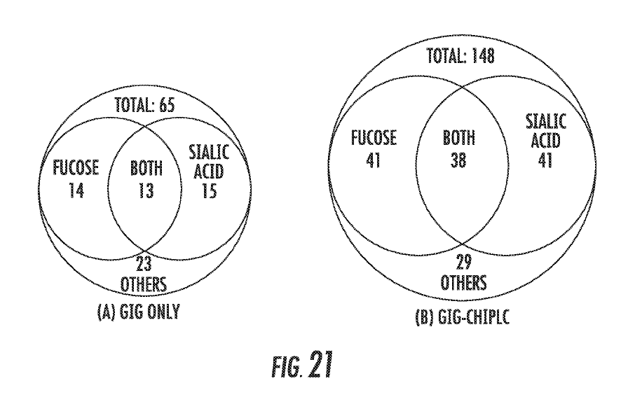

Glycomic analysis of complex biological samples by GIG and MS showed increased glycan coverage by detecting low abundant glycans. When GIG was applied to the analysis of N-glycans from human serum, we detected 65 N-glycan masses without glycan fractionation (FIG. 21A and Table 7). Over 40% of the detected 65 N-glycan masses were sialylated in which 13 N-glycans were also fucosylated. When GIG-chip-LC was used to analyze N-glycans isolated from human serum with chip-LC fraction, the number of N-glycan masses detected by MALDI-MS increased to 148 (FIG. 21B and Table 7). We detected 79 sialylated glycan masses and an additional 41 fucosylated glycan masses. The increased number of N-glycans observed after chip-LC separation could be attributed to reduced dynamic range of glycan concentrations in each fraction. As a result, low-abundance glycans were effectively eluted in a specific fraction by graphitized carbon and detected by MS without the ion competition of other high abundant glycan ions.

TABLE 7
Detected N-glycan masses and proposed glycan composition from
human serum by GIG with and without chipLC separation. Glycans
were detected by MALDI-MS using Shimadzu AXIMA Resonance.

| | | | | | | | | Eluted |
|---|---|---|---|---|---|---|---|---|
| | | | | | m/z | Intensity | | in No of |
| List | Fucose | HexNAc | Hexose | NeuAc | (Da) | No LC | chipLC | Fraction |
| 1 | 0 | 0 | 0 | 0 | 933.1 | 3123 | 5320 | 1 |
| 2 | 1 | 0 | 0 | 0 | 1079.1 | 2103 | 4302 | 2 |
| 3 | 0 | 0 | 1 | 0 | 1095.2 | 1503 | 3058 | 2 |
| 4 | 0 | 1 | 0 | 0 | 1136.2 | 3258 | 703 | 1 |
| 5 | 0 | 0 | 2 | 0 | 1257.1 | 5463 | 7653 | 3 |
| 6 | 1 | 1 | 0 | 0 | 1282.2 | 3501 | 3750 | 2 |
| 7 | 0 | 1 | 1 | 0 | 1298.2 | 6661 | 1716 | 3 |
| 8 | 0 | 2 | 0 | 0 | 1339.2 | 2578 | 3208 | 3 |
| 9 | 1 | 0 | 2 | 0 | 1403.1 | 445 | 350 | 1 |
| 10 | 0 | 0 | 3 | 0 | 1419.2 | 5213 | 8307 | 3 |
| 11 | 1 | 1 | 1 | 0 | 1444.2 | 2301 | 1148 | 1 |
| 12 | 0 | 1 | 2 | 0 | 1460.3 | 1742 | 1802 | 1 |
| 13 | 1 | 2 | 0 | 0 | 1485.2 | 15860 | 36922 | 2 |
| 14 | 0 | 2 | 1 | 0 | 1501.2 | 2407 | 2484 | 2 |
| 15 | 0 | 1 | 0 | 1 | 1516.1 | 2983 | 1203 | 2 |
| 16 | 0 | 3 | 0 | 0 | 1542.1 | 4581 | 11822 | 2 |
| 17 | 0 | 0 | 4 | 0 | 1581.2 | 1416 | 3222 | 2 |
| 18 | 0 | 1 | 3 | 0 | 1622.2 | 902 | 1754 | 1 |
| 19 | 1 | 2 | 1 | 0 | 1647.2 | 10422 | 21198 | 2 |
| 20 | 0 | 2 | 2 | 0 | 1663.2 | 442 | 1790 | 2 |
| 21 | 0 | 1 | 1 | 1 | 1678 | 7105 | 3500 | 2 |
| 22 | 1 | 3 | 0 | 0 | 1688.2 | 2472 | 10539 | 1 |
| 23 | 0 | 3 | 1 | 0 | 1704.3 | 2024 | 3983 | 2 |
| 24 | 0 | 0 | 5 | 0 | 1743.3 | 1595 | 3206 | 2 |
| 25 | 2 | 2 | 1 | 0 | 1791.2 | | 407 | 1 |
| 26 | 1 | 2 | 2 | 0 | 1809.2 | 3155 | 2165 | 7 |
| 27 | 0 | 2 | 3 | 0 | 1825.2 | 1461 | 2605 | 7 |
| 28 | 1 | 1 | 1 | 0 | 1840.5 | 985 | 715 | 5 |
| 29 | 1 | 3 | 1 | 0 | 1850.3 | 2253 | 5193 | 2 |
| 30 | 0 | 3 | 2 | 0 | 1866.3 | 723 | 1719 | 2 |
| 31 | 0 | 2 | 1 | 1 | 1882.2 | 1670 | 956 | 8 |
| 32 | 0 | 0 | 6 | 0 | 1905.2 | 1850 | 1595 | 5 |
| 33 | 3 | 2 | 1 | 0 | 1942.1 | | 1267 | 6 |
| 34 | 1 | 0 | 3 | 1 | 1945.2 | | 481 | 1 |
| 35 | 3 | 3 | 0 | 0 | 1979.2 | | 446 | 1 |
| 36 | 1 | 1 | 2 | 1 | 1984.2 | | 534 | 2 |
| 37 | 2 | 3 | 1 | 0 | 1995.3 | | 988 | 1 |
| 38 | 0 | 1 | 3 | 1 | 2002.2 | | 685 | 1 |
| 39 | 1 | 3 | 2 | 0 | 2012.2 | 527 | 2993 | 3 |
| 40 | 1 | 2 | 1 | 1 | 2027.2 | | 3125 | 4 |
| 41 | 0 | 3 | 3 | 0 | 2028.3 | 420 | 1593 | 3 |
| 42 | 0 | 2 | 2 | 1 | 2043.2 | 16833 | 8646 | 22 |
| 43 | | | | | 2053.2 | 1190 | 2035 | 3 |
| 44 | 0 | 1 | 1 | 2 | 2059.3 | | 1022 | 1 |
| 45 | 0 | 4 | 2 | 0 | 2068.2 | | 841 | 1 |
| 46 | 0 | 3 | 1 | 1 | 2084.2 | | 4460 | 3 |
| 47 | 0 | 1 | 6 | 0 | 2108.2 | | 678 | 1 |
| 48 | 2 | 2 | 3 | 0 | 2119.2 | | 376 | 1 |
| 49 | 1 | 2 | 4 | 0 | 2133 | | 483 | 1 |
| 50 | 3 | 3 | 1 | 0 | 2145.1 | | 1275 | 1 |
| 51 | 1 | 3 | 3 | 0 | 2174.3 | 540 | 1220 | 3 |
| 52 | 0 | 0 | 3 | 2 | 2179.2 | | 1483 | 2 |
| 53 | 3 | 4 | 0 | 0 | 2187.2 | | 1307 | 2 |
| 54 | 1 | 2 | 2 | 1 | 2189.3 | 4179 | 1504 | 9 |
| 55 | 0 | 3 | 4 | 0 | 2190.2 | | 2554 | 5 |
| 56 | 0 | 0 | 5 | 1 | 2202.2 | | 3175 | 5 |
| 57 | 0 | 2 | 3 | 1 | 2205.3 | | 1059 | 1 |
| 58 | 3 | 1 | 4 | 0 | 2221.1 | | 505 | 2 |
| 59 | 1 | 3 | 1 | 1 | 2230.2 | 579 | 7744 | 1 |
| 60 | 0 | 4 | 3 | 0 | 2231.2 | 402 | 2018 | 2 |
| 61 | 0 | 3 | 2 | 1 | 2246.2 | 946 | 9922 | 2 |
| 62 | 4 | 2 | 2 | 0 | 2248.1 | | 1239 | 5 |
| 63 | 0 | 2 | 1 | 2 | 2261.2 | | 536 | 1 |
| 64 | 4 | 1 | 1 | 1 | 2262.3 | | 606 | 3 |
| 65 | 3 | 2 | 3 | 0 | 2263 | | 1246 | 5 |
| 66 | 0 | 0 | 6 | 1 | 2285.2 | 1031 | 2695 | 2 |
| 67 | 0 | 4 | 1 | 1 | 2291.1 | | 1453 | 2 |
| 68 | 3 | 3 | 2 | 0 | 2306.2 | | 577 | 2 |
| 69 | 1 | 1 | 4 | 1 | 2309.3 | | 719 | 3 |
| 70 | 2 | 3 | 3 | 0 | 2320.2 | 2733 | 3612 | 5 |
| 71 | 0 | 1 | 5 | 1 | 2326.3 | | 883 | 1 |
| 72 | 2 | 2 | 2 | 1 | 2335.2 | 303 | 1023 | 2 |
| 73 | 1 | 1 | 2 | 2 | 2364.1 | | 656 | 2 |
| 74 | 0 | 2 | 4 | 1 | 2365.2 | | 510 | 1 |
| 75 | 1 | 4 | 3 | 0 | 2377.2 | 502 | 1554 | 2 |
| 76 | 0 | 1 | 3 | 2 | 2382.2 | | 607 | 1 |
| 77 | 0 | 0 | 9 | 0 | 2391.2 | | 714 | 3 |
| 78 | 2 | 2 | 0 | 2 | 2392.3 | | 1570 | 2 |
| 79 | 1 | 3 | 2 | 1 | 2393.2 | 2976 | 21793 | 2 |
| 80 | 0 | 4 | 4 | 0 | 2393.2 | 1006 | 2790 | 2 |
| 81 | 1 | 2 | 1 | 2 | 2408.9 | | 816 | 4 |
| 82 | 0 | 3 | 3 | 1 | 2409.9 | 3032 | 2550 | 11 |
| 83 | 0 | 2 | 2 | 2 | 2424.3 | 52646 | 28889 | 16 |
| 84 | 3 | 1 | 3 | 1 | 2440 | | 2100 | 2 |
| 85 | 0 | 4 | 2 | 1 | 2451.2 | 350 | 561 | 1 |
| 86 | 3 | 3 | 3 | 0 | 2466.9 | | 819 | 1 |
| 87 | 0 | 5 | 1 | 1 | 2490.9 | | 2722 | 1 |
| 88 | 2 | 2 | 3 | 1 | 2498.8 | | 759 | 1 |
| 89 | 0 | 2 | 5 | 1 | 2529.8 | | 1228 | 1 |
| 90 | 2 | 2 | 1 | 2 | 2554.9 | | 580 | 2 |
| 91 | 1 | 3 | 3 | 1 | 2555.9 | 1528 | 1078 | 7 |
| 92 | 0 | 8 | 0 | 0 | 2557.9 | | 398 | 1 |
| 93 | 0 | 0 | 3 | 3 | 2560.9 | | 3792 | 3 |
| 94 | 1 | 2 | 2 | 2 | 2570.9 | 6983 | 5019 | 12 |
| 95 | 0 | 2 | 3 | 2 | 2586.9 | | 497 | 1 |
| 96 | 0 | 5 | 4 | 0 | 2597.3 | 300 | 602 | |
| 97 | 0 | 4 | 3 | 1 | 2609.9 | | 546 | 1 |
| 98 | 0 | 3 | 2 | 2 | 2627.8 | 527 | 1199 | 6 |
| 99 | 3 | 3 | 4 | 0 | 2628.9 | | 1902 | 1 |
| 100 | 2 | 7 | 0 | 0 | 2647 | | 203 | 1 |
| 101 | 1 | 7 | 1 | 0 | 2650.9 | | 940 | 1 |
| 102 | 3 | 4 | 3 | 0 | 2672.6 | | 815 | 4 |
| 103 | 2 | 4 | 4 | 0 | 2687.4 | | 1154 | 2 |
| 104 | 1 | 8 | 0 | 0 | 2703.2 | | 540 | 1 |
| 105 | 1 | 0 | 8 | 1 | 2755.9 | | 1079 | 2 |
| 106 | 1 | 4 | 3 | 1 | 2758.9 | | 504 | 1 |
| 107 | 0 | 0 | 9 | 1 | 2771.9 | | 1447 | 3 |
| 108 | 1 | 3 | 2 | 2 | 2773.9 | 3854 | 10581 | 4 |
| 109 | 0 | 4 | 4 | 1 | 2774.7 | 350 | 573 | 5 |
| 110 | 0 | 3 | 3 | 2 | 2789.9 | 4906 | 4736 | 15 |
| 111 | 4 | 2 | 3 | 1 | 2790.7 | | 685 | 5 |
| 112 | 2 | 8 | 0 | 0 | 2849.7 | | 424 | 1 |
| 113 | 0 | 2 | 5 | 2 | 2909.9 | | 601 | 2 |
| 114 | 1 | 4 | 4 | 1 | 2920.5 | | 328 | 2 |
| 115 | 1 | 3 | 3 | 2 | 2935.3 | 2882 | 1615 | 12 |
| 116 | 4 | 5 | 1 | 0 | 2937 | | 679 | 2 |
| 117 | 1 | 2 | 2 | 3 | 2949 | | 1215 | 3 |
| 118 | 0 | 3 | 4 | 2 | 2950 | | 848 | 2 |
| 119 | 4 | 2 | 2 | 2 | 3008 | | 269 | 1 |
| 120 | 2 | 4 | 6 | 0 | 3010 | | 817 | 1 |
| 121 | 1 | 4 | 7 | 0 | 3028.1 | | 301 | 1 |
| 122 | 0 | 5 | 2 | 2 | 3032 | | 1029 | 3 |
| 123 | 2 | 5 | 5 | 0 | 3053 | | 568 | 1 |
| 124 | 2 | 4 | 4 | 1 | 3068.8 | | 1162 | 4 |
| 125 | 1 | 3 | 2 | 3 | 3154.2 | 829 | 1103 | 1 |
| 126 | 0 | 4 | 4 | 2 | 3154.2 | | 1346 | 3 |
| 127 | 3 | 4 | 6 | 0 | 3155.2 | | 663 | 10 |
| 128 | 0 | 3 | 3 | 3 | 3170.1 | 6816 | 7468 | 13 |
| 129 | 3 | 3 | 5 | 1 | 3171.3 | | 680 | 2 |
| 130 | 2 | 8 | 2 | 0 | 3174.1 | | 728 | 1 |
| 131 | 1 | 5 | 2 | 2 | 3178.1 | | 453 | 1 |
| 132 | 0 | 5 | 6 | 1 | 3301.6 | | 598 | 2 |
| 133 | 1 | 3 | 3 | 3 | 3316.2 | 2015 | 3889 | 11 |
| 134 | 2 | 4 | 4 | 2 | 3447.1 | | 212 | 1 |
| 135 | 2 | 3 | 3 | 3 | 3462.1 | | 597 | 3 |
| 136 | 1 | 4 | 3 | 3 | 3519.1 | | 229 | 1 |
| 137 | 0 | 4 | 4 | 3 | 3535.1 | 817 | 1291 | 13 |
| 138 | 1 | 4 | 4 | 3 | 3681.2 | 465 | 478 | 12 |
| 139 | 0 | 3 | 4 | 4 | 3712.8 | | 168 | 1 |
| 140 | 0 | 6 | 3 | 3 | 3776.2 | | 414 | 2 |
| 141 | 2 | 4 | 4 | 3 | 3827.2 | | 303 | 4 |
| 142 | 0 | 5 | 5 | 3 | 3901.3 | | 2611 | 2 |
| 143 | 0 | 4 | 4 | 4 | 3915.7 | 533 | 1635 | 7 |
| 144 | 3 | 4 | 4 | 3 | 3974.4 | | 191 | 1 |
| 145 | 1 | 4 | 4 | 4 | 4061.3 | 299 | 642 | 8 |
| 146 | 2 | 4 | 4 | 4 | 4207.3 | 120 | 216 | 1 |
| 147 | 0 | 5 | 5 | 4 | 4281.3 | | 207 | 2 |
| 148 | 3 | 4 | 4 | 4 | 4355.4 | | 239 | 1 |

The number of N-glycan structures could be much larger than the 148 N-glycan mass peaks detected from serum (FIG. 21B). Each N-glycan mass can potentially correspond to a number of isomers, sometimes over a dozen for a single mass. Importantly, each isomer may have its own physical and biological properties. Different isomers can further be distinguished by glycan permethylation and tandem MS. Permethylation of glycans can be implemented in the microfluidic system by packing sodium hydroxide particles in a microchip platform. Capillary permethylation has been a recent rising technology for glycan derivatization. In an embodiment, the extracted glycans from GIG can be infused into sodium hydroxide-packed microchip for glycan permethylation, followed by chip-LC profiling. Thus, this innovative platform should be beneficial for glycomics analysis by providing linkage information corresponding to assigned glycan structures.

The use of the integrated microchip for glycan isolation and separation described in the present invention demonstrates several advantages over traditional chromatography. In addition to advantages of microchip for solid-phase glycan extraction, the mesoporous PGC used for glycan separation has remarkably high surface area. The PGC particle size is around 45 μm in our study. The surface area in a single separation channel was estimated as following: The channel cross section is 800 μm×800 μm and length is 20 mm. Thus, the total separation channel volume equals 0.0128 cm³; the density of carbon particles is 0.5 cm³/g, thus a total of 25.6 mg particles can be packed in separation channel; the estimated surface area of packed carbon particles is 6.4 m² based on its specific surface area. The commercial PGC, e.g., Hypercarb made by Thermo Scientific, has an average pore size of 250 Å and the specific surface area of 120 m²/g, that is 3.1 m² for same volume of particles. Subsequently, longer commercial columns are needed to achieve same surface area of the fabricated microchip, resulting in increased back-pressure during separation. In addition, the microchip of the present invention is low-cost, and separation particles can be re-packed without sacrificing separation performance.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of isolating glycans from glycoproteins in a biological sample comprising:
a) obtaining a biological sample comprising glycoproteins;
b) denaturing the sample of a) to denature the glycoproteins and/or glycopeptides;
c) conjugating the denatured glycoproteins and/or glycopeptides from b) with reductive amination to aldehyde groups on solid support;
d) blocking unreacted aldehyde groups on the solid support with reductive amination;
e) removing unconjugated glycoproteins and/or glycopeptides and remaining components;
f) releasing the glycans from the glycoproteins and/or glycopeptides bound to the solid support of c); and
g) isolating the glycans released from f).

2. The method of claim 1, further comprising h) analyzing the glycans of g).

3. A method of isolating glycans in a biological sample comprising:
a) obtaining a biological sample comprising glycoproteins;
b) denaturing the sample of a) to denature the glycoproteins and/or glycopeptides;
c) reacting the glycoproteins and/or glycopeptides of b) with guanidine to convert lysine to homoarginine;
d) conjugating the denatured glycoproteins and/or glycopeptides from b) with reductive amination to aldehyde groups on solid support;
e) blocking unreacted aldehyde groups on the solid support with reductive amination;
f) labeling aspartic acid groups by aniline using isotopes;
g) performing an Asp-N digest to remove any unlabeled aspartic acid residues;
h) releasing the N-glycans from the glycoproteins and/or glycopeptides bound to the solid support of c) using PNGase F;
i) digesting glycoproteins and/or glycopeptides on beads with Asp-N to release N-glycopeptides at the N-terminal of the glycosylation motif (▾NXT/S);
j) isolating the isolated glycans released from h); and
k) analyzing the glycans of g).

4. A method for isolating sialylated glycans in a biological sample comprising:
a) obtaining a biological sample comprising glycoproteins;
b) denaturing the sample of a) to denature the glycoproteins and/or glycopeptides;
c) conjugating the denatured glycoproteins and/or glycopeptides from b) with reductive amination to aldehyde groups on solid support;
d) blocking unreacted aldehyde groups on the solid support with reductive amination;
e) removing unconjugated glycoproteins and/or glycopeptides and remaining components;
f) derivitizing denatured glycoproteins and/or glycopeptides of c) with p-toluidine;
g) releasing the glycans from the glycoproteins and/or glycopeptides bound to the solid support of c); and
h) isolating the glycans released from g).

5. The method of claim 4, further comprising i) analyzing the glycans of h).

6. A method for determining the number of sialic acid residues on isolated sialylated glycans in a biological sample comprising:
a) obtaining a biological sample comprising glycoproteins;
b) denaturing the sample of a) to denature the glycoproteins and/or glycopeptides;
b1) dividing the denatured sample of b) into two or more aliquots;
c) conjugating each aliquot of the denatured glycoproteins and/or glycopeptides from b) with reductive amination to aldehyde groups on solid support;
d) blocking unreacted aldehyde groups on the solid support with reductive amination;
e) removing unconjugated glycoproteins and/or glycopeptides and remaining components;
f) derivitizing at least one aliquot of the denatured glycoproteins and/or glycopeptides of c) with light p-toluidine, and derivitizing at least one other aliquot of the denatured glycoproteins and/or glycopeptides of c) with heavy p-toluidine;
g) releasing the glycans from each aliquot of the glycoproteins and/or glycopeptides bound to the solid support of c); and
h) isolating the glycans released from each aliquot of g).

7. The method of claim 6, further comprising i) analyzing the glycans of h).

8. The method of claim 1, wherein in the conjugation of c) is performed using aniline.

9. The method of claim 1, wherein the biological sample is from a subject.

10. The method of claim 9, wherein the step of analyzing is performed using an analytical method selected from the group consisting of MS, HPLC, and CE.

11-13. (canceled)

14. An apparatus for analysis of glycans in a sample comprising:
a) a substrate in the form of a chip having at least a first and second layer, wherein the first layer is a fluid layer having at least a first and second channel, each channel having an inlet and an outlet and wherein each of the channels having a separation portion and a constrained portion, and wherein the first channel comprises a stationary phase for liquid chromatographic separation of glycans, and wherein the second channel comprises an aldehyde activated agarose bead resin, the outlet of the second channel intersects with the first channel and communicates with the first channel at a position proximal to the inlet of the first channel;
b) the second layer is a coverslip layer which is fitted over top of the fluid layer and has at least three reservoirs, each having a removable cap which closes access to the inlet or outlet, wherein the first reservoir communicates with inlet of the first channel, the second reservoir communicates with inlet of the second channel, and the third reservoir communicates with outlet of the first channel; and
c) the second layer is bonded to the first layer to make a liquid seal.

15. The apparatus of claim 14, wherein the substrate is a polymer.

16. The apparatus of claim 14, wherein the substrate is a cyclic olefin polymer.

17. The apparatus of claim 14, wherein the separation portion of the first or second channel has dimensions of 800 μm×800 μm.

18. The apparatus of claim 14, wherein the constrained portion of the first or second channel has dimensions of 50 μm×50 μm.

19. The apparatus of claim 14, wherein the at least three reservoirs have an opening capable of fitting a 22 gauge steel needle.

20. The apparatus of claim 14, wherein the stationary phase for liquid chromatographic separation of glycans comprises porous graphitized carbons (PGC).

21. The apparatus of claim 14, wherein the aldehyde activated agarose bead resin is AminoLink™ resin.

22. A method for isolating glycans in a sample comprising:
a) injecting a sample containing glycans into the inlet of the second channel of the apparatus of claim 14 with the inlet of the first channel open and the outlet of the first channel closed;
b) conjugating any proteins in the sample to the aldehyde activated agarose bead resin in the second channel;
c) reducing the conjugated proteins of b) with a reducing reagent and blocking any free aldehyde groups on the resin in the second channel;
d) washing the second channel with water;
e) releasing the glycans of c) with a releasing agent in the second channel;
f) flushing the glycans of e) via the outlet of the second channel into the first channel at a position proximal to the inlet of the first channel;
g) closing the inlet of the second channel and pumping mobile phase into the inlet of the first channel while collecting eluent containing the released glycans of f) from the outlet of the first channel.

23. The method of claim 22, further comprising h) analyzing the glycans in the eluent.

24. The method of claim 22, wherein the sample is a biological sample is from a subject.

25. The method of claim 23, wherein the step of analyzing is performed using an analytical method is selected from the group consisting of MS, HPLC, and CE.

26. The method of claim 25, wherein the analytical method is MALDI-MS.

27-29. (canceled)
